SEQUENCE LISTING (9 sequences)

SEQ ID NO: 1 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 2 - protein, 255 aa, Rattus norvegicus
MAHLMAGQWLLLLMWMAECAQSRATRARTELLNVCMDAKHHKEKPGPEDKLHDQCSPWKTNACCSTNTSQEAHKDISYLYRFNWNHCGTMTPECKRHFIQDTCLYECSPNLGPWIQQVDQSWRKERILDVPLCKEDCVLWWEDCKSSFTCKSNWHKGWNWTSGHNECPVGASCHPFTFYFPTPAVLCEKIWSHSYKLSNYSRGSGRCIQMWFDPAQGNPNEEVARFYAEVMSGAGLREAWLLVCSLSLVLFWVLS

SEQ ID NO: 3 - protein, 251 aa, Capra hircus
MAWKITPLLCLLVCVAAVGAARPRTQLLNVCMNARYHKEKPGPEDKLHGQCSPWKNNACCFVNTSIEAHKDISSLYRFDWDHCGKMEPACKRHFIQDICLYECSPNLGPWIQEVNQSWRKERILNVPLCKEDCESWWEDCRTSYTCKSNWHRGWDWTSGYNQCPVKVACHRFDFYFPTPAALCNEIWSHSYRASNYSRGSGRCIQMWFDPVLGNPNEEVARFYAESMNGAGLHEAWPLRFGLLLLLLWLLS

SEQ ID NO: 4 - protein, 251 aa, Bos taurus
MAWKITPLLCLLVCVAAVGAARPGTQLLNVCMNARYHKEKPGPEDKLHGQCSPWKKNACCFVNTSIEAHKDISSLYRFDWDHCGKMEPACKRHFIQDTCLYECSPNLGPWIREVNQSWRKERILNVPLCKEDCQSWWEDCRTSYTCKSNWHRGWDWTSGYNQCPVKAACHRFDFYFPTPAALCNEIWSHSYKASNYSRGSGRCIQMWFDPILDNPNEEVARFYAESMNGAGLHEAWPLRCGLLLLLLWLLS

SEQ ID NO: 5 - protein, 257 aa, Homo sapiens; identical to SEQ ID NO: 1

SEQ ID NO: 6 - protein, 203 aa, Rattus norvegicus
MAHLMAGQWLLLLMWMAECAQSRATRARTELLNVCMDAKHHKEKPGPEDKLHDQCSPWKTNACCSTNTSQEAHKDISYLYRFNWNHCGTMTPECKRHFIQDTCLYECSPNLGPWIQQVDQSWRKERILDVPLCKEDCVLWWEDCKSSFTCKSNWHKGWNWTSGHNECPVGASCHPFTFYFPTPAVLCEKIWSHSYKLSNYSRG

SEQ ID NO: 7 - protein, 63 aa, Capra hircus
CSPWKNNACCFVNTSIEAHKDISSLYRFDWDHCGKMEPACKRHFIQDICLYECSPNLGPWIQE

SEQ ID NO: 8 - protein, 253 aa, Capra hircus
MPWKLTPLLLFLGWMTSVCNARTRTDLLNVCMDAKHHKAEPGPEDKLHNQCTPWKKNACCSARVSQELHKDTSSLYNFTWDHCGKMEPACQRHFIQDNCLYECSPNLGPWIQEVNQKWRKERFLNVPLCKEDCQSWWEDCRTSHTCKSNWHRGWDWTSGSNKCPNGTTCRTFEAYFPTPAALCEGLWSHSYKLSNYSRGSGRCIQMWFDPALGNPNEEVARFYASALTAEAWPQGIRPLSLCLALMLSLWLHD

SEQ ID NO: 9 - DNA, 990 bp, Bos taurus
  source 1..990: mol_type = other DNA
ccactaagcccttctgtcccctagagtgaggcaagggggactgcagagcagaacagaagcctgagccagacgaacagccagcattcctcccaggaaattggttcttcagagacaggcatggcctggaagatcacaccactgctgtgccttttagtgtgtgtggctgctgtgggggcagcccggcccgggactcagcttctcaacgtctgcatgaatgccaggtaccataaggaaaaaccaggtcccgaggacaagttacatggacagtgcagcccttggaaaaagaatgcctgctgctttgtcaacaccagcatagaagcccataaggatatttccagcctgtacagattcgactgggaccactgcggcaagatggagcctgcatgcaagcgccactttattcaggacacctgtctctacgagtgctcacctaatctggggccctggatccgggaggtgaatcagagctggcgcaaagaacggatcctgaatgtgcccctctgcaaagaggactgtcagagctggtgggaagactgccgcacctcctacacctgcaagagcaactggcacaggggctgggactggacctcagggtacaaccagtgcccagtgaaagctgcctgccaccgcttcgacttctacttcccgacgcctgctgctctgtgcaatgaaatctggagtcactcctacaaagccagcaactacagtcgtggcagcggccgctgcattcagatgtggttcgaccccatcctggacaaccccaacgaggaggtggcgagattctatgctgagagcatgaatggggctgggctccatgaggcctggcctctgcgttgcggcctgctcttattgttactctggctgctcagttgagctcctattattttttgacacctggaaatccctgccccgttaagctccacagtcagtttgttccttggtggaaactgtttgaataaaaagtcaatcaccctaaaaaaaaaaaaaaaa

(19) United States
(12) Patent Application Publication
(10) Pub. No.: US 2023/0172222 A1
(43) Pub. Date: Jun. 8, 2023

(54) REDEFINING LIVESTOCK MILK BY REMOVING FOLATE RECEPTOR PROTEIN TO DECREASE FOLATE RECEPTOR AUTOIMMUNE DISORDER IN PREGNANCY AND AUTISM

(71) Applicant: Miritaur LLC, Brooklyn, NY (US)

(72) Inventors: Prem Premsrirut, Brooklyn, NY (US); Edward V. Quadros, Brooklyn, NY (US)

(21) Appl. No.: 17/871,073

(22) Filed: Jul. 22, 2022

Related U.S. Application Data
(63) Continuation of application No. PCT/US2021/014720, filed on Jan. 22, 2021.
(60) Provisional application No. 62/964,343, filed on Jan. 22, 2020.

(51) Int. Cl. A23C 9/148; C07K 1/22; C07K 1/16; C12N 15/85; C12N 15/113; B01D 15/38; B01D 15/34

(52) U.S. Cl. CPC A23C 9/148; C07K 1/22; C07K 1/16; C12N 15/8509; C12N 15/1138; B01D 15/3809; B01D 15/34; B01D 15/3828; C12N 2830/008; B01D 2015/3838

(57) ABSTRACT

Provided herein are systems and methods in Redefining Cow Milk to decrease the prevalence of folate receptor autoimmune disorder in the population to reduce the Incidence of neural tube defect pregnancy, pre-term birth, subfertility and a plethora of developmental disorders including autism spectrum disorders.

### CROSS-REFERENCE TO RELATED APPLICATIONS

The present application claims priority from PCT application serial no. PCT/US2021/014720 filed Jan. 22, 2021, which claim priority from U. S provisional application Ser. No. 62/964,343 filed Jan. 22, 2020, all herein incorporated by reference.

### SEQUENCE LISTING

The present application contains a Sequence Listing which is submitted electronically in XML format and is incorporated by reference in its entirety. Said XML file was created on Jan. 23, 2023, is named XML Sequence MIS-101-US-CON.xml and 11,593 bytes in size.

### FIELD OF THE INVENTION

The invention relates to the use of milk free of folate receptor (FR). Presence of the folate receptor in milk can induce the formation of autoantibodies against the human folate receptor which can cause folate-sensitive abnormalities, such as birth defects (e.g., a neural tube defect, i.e. NTD), infertility, spontaneous abortion, male sterility, unsuccessful in vitro fertilization, neurological disorders, neurodevelopmental delays and neuropsychiatric diseases, such as cerebral folate deficiency or autism spectrum disorder. The invention relates to generating milk (from cattle, goat, and other livestock used to produce milk for human consumption) whereby folate receptor has been removed by extraction or generated from engineered animals in which expression of the folate receptor is silenced or inhibited in the mammary gland, thus preventing production and secretion into milk.

### BACKGROUND

The invention generally relates to the production of folate receptor free (FRF) milk and milk products. Exposure to nonhuman milk or milk products containing nonhuman folate receptor can illicit autoantibodies against the human folate receptor/folate binding protein (FR/FBP) which can block folate or folic acid transport from the mother to the fetus and to the brain in infants (Rothenberg et al, 2004; Ramaekers et al 2005).

Folic acid is essential for normal embryonic development because it participates in one-carbon metabolism for the synthesis of nucleic acids and amino acids required by highly proliferative embryonic cells (Lucock, Mal. Genet. Metab. 71:121 (2000)). Maternal nutrition has linked folate intake to the prevention of NTDs (Hibbard and Smithells, Obstet. Gynaecol. Br. Commonwealth 71:529 (1964); Hibbard and Smithells, Lancet 1, 1254(1965); Smithall et al., Arch. Dis. Child 51:944 (1976)) and supplementation of folate during pregnancy is now common practice. However, many cases of NTDs or other birth anomalies occurred in mothers who did not exhibit signs of clinical folate deficiency, leading researchers to identify other causes that impair cellular metabolism or uptake of folate. While many genetic causes have been identified, these genes account for only a small number of birth defects (van der Put et al., Exp. Biol. Med. (Maywood), 226:243 (2001)).

In a prior patent publication (U.S. Pat. No. 7,846,672), Dr. Edward Quadros describe the discovery of folate-sensitive disorders, such as infertility, spontaneous abortion, unsuccessful in vitro fertilization, or birth defects, that are due to interference of folate uptake by an autoantibody against the folate receptor. In addition, they describe assays to detect these autoantibodies to the folate receptor.

Subsequent studies revealed that over 70% of children with autism spectrum disorder (ASD) are positive for the FR antibody (Frye et al., Mol. Psych. 18:369-381, (2013)). These antibodies can block folate transport to the brain. Notably, we show that these FR antibodies react strongly with FR from cow (bovine) milk (Ramaekers et al. 2009). Cow (bovine) milk contains substantial amounts (1-3 ug/ml) of folate receptor alpha [FR], which belongs to a group of Folate Binding Proteins [FBP] involved in folate transport. Since human and bovine FR are structurally similar, molecular mimicry likely accounts for the cross reactivity.

Many reports show that children with ASD are more likely to have been either bottle fed or weaned early than comparison groups not affected by ASD. This suggests that early introduction to bovine or nonhuman mammalian milk may increase the risk for ASD and related neurological deficits. Intake of conventional bovine milk during early development has been implicated in contributing to or causing autism and other neurological deficits. This is believed to be related to disruption of folate metabolism since folate is an essential nutrient during fetal and infant development. In order to introduce more dietary folate, several “folate enhanced” milk products have been introduced, although their benefit has not been proven.

Recent published work by Dr. Edward Quadros provides clear evidence that folate receptor autoantibodies are prevalent in children diagnosed with autism spectrum disorder, their normal siblings and parents (Ramaekers et al., Mol Psych. 18:270-271 (2013); Frye et al., Mol Psysch. 18:369-381, (2013)). Intervention strategies using high dose folinic acid treatment in children with ASD who also express FR autoantibodies have proven beneficial for learning deficits and behavioral improvements (Frye et al., Mol. Psych. 23:247-256, (2018)). Further multi-center clinical trials are underway.

Although high dose folinic acid offers a therapeutic treatment to children with cognitive deficits and FR antibodies, it does not remove the insult that triggers autoantibody formation against the FR. In children positive for FR antibody, a milk-free diet decreases antibody titer (Ramaekers et al. 2009), but this diet is extremely restrictive and difficult to comply with.

Autism Spectrum Disorder is a developmental disorder that affects communication, social interaction and behavior. Symptoms of ASD are usually evident after the first two years of life and are characterized by: Difficulty with communication and interaction with other people; Restricted interests and repetitive behaviors; Symptoms that hurt the person's ability to function properly in school, work and other areas of life. Autism is a “spectrum” because there is wide variation in type and severity of symptoms people experience. CDC estimates 1 in 59 individuals in the US (˜1-2%) are affected by ASD. Although rates in China are fully recorded, an estimated 1 in 100 children may be affected (Wang et al. Int J Biol Sci 2018; 14(7): 717-725). In most parts of the world, this varies between 1 in 55 to 1 in 200.

There are no known causes of ASD, although genetic and environmental factors have been suggested as associated with ASD.

Food allergies are more prevalent in children with ASD where 11.25% have allergies compared to 4.25% in a study of ˜200,000 children. Autoimmune conditions are also more frequently reported among children. A new “fragile gut” hypothesis describes how some children with impaired gut absorption may make autoantibodies to their folate receptor and this may lead to symptoms of ASD. Folate is essential for fetal and neonatal neurodevelopment and disruption of folate metabolism has recently been identified in ASD patients.

A normal robust gut is shown in FIG. 1A. Healthy digestion of proteins and simple sugars occurs in the small intestine, making nutrients absorbable. Fiber and undigested proteins are consumed by bacteria. Blood does NOT interact with the lumen of the intestine, preventing any interaction between immune cells and antigenic food molecules.

A fragile gut is shown in FIG. 1B. Children with ASD display reduced digestive capacity. Inflammation and deterioration of the gut lining prevents proper digestion of proteins and sugars. Inflammation leads to direct contact of blood with the intestine, allowing undigested proteins in the intestine to pass DIRECTLY into the blood stream and or get exposed to GALT (gut associated lymphatics) and MALT (mucosal associated lymphatics). This allows for unwanted exposure to antigenic proteins within the immune tissue to provoke an immune response.

A fragile gut may lead to autoantibodies to cow milk proteins, including the folate receptor, which is secreted in cow milk (1-2 ug/ml). Anti-folate receptor antibodies isolated from children with ASD have been shown to cross-react strongly with bovine folate receptors in cow milk. Antibody concentration is significantly reduced when children are placed on a milk-free diet. Milk-free diets are hard to comply with.

Folate is required for many processes during brain development. Folate metabolism is shown in FIG. 2 and is often abnormal in ASD patients. 70% of children with ASD have autoantibodies that block folate receptors and prevent folate uptake in cells (Frye, R. E. et al. Cerebral folate receptor autoantibodies in autism spectrum disorder. Mol Psych, 2013). Exposure to folate receptor antibodies in pregnant rats during gestation and in pups during weaning causes severe behavioral deficits (Sequiera, J. M. et al. Plos one, 2016).

Folate Deficiency can be corrected with Folinic acid, a reduced form of folate, that can enter the brain via the choroid plexus, independent of the folate receptor. Folate Deficiency may be corrected with high dose folinic acid that does not repair the damage, but can restore cerebral folate to treat symptoms of ASD. Folate Deficiency may be corrected by restoring brain folate, which produces clinical improvement in language and social communication in ASD children.

The present invention is designed to solve the cause of folate receptor autoimmune disorder and preventing its pathologic consequences.

### SUMMARY OF THE INVENTION

Provided herein are assays and methods to produce folate receptor free (FRF) milk from a mammalian source. The methods and systems are set forth in part in the description which follows, and in part will be obvious from the description, or can be learned by practice of the methods, apparatuses, and systems. The advantages of the methods and systems will be realized and attained by means of the elements and combinations particularly pointed out in the appended claims. It is to be understood that both the foregoing general description and the following detailed description are exemplary and explanatory only and are not restrictive of the methods and systems, as claimed.

### BRIEF DESCRIPTION OF THE DRAWINGS

In the accompanying figures, like elements are identified by like reference numerals among the several preferred embodiments of the present invention.

FIG. 1A is a schematic diagram showing a normal robust gut.

FIG. 1B is a schematic diagram showing a fragile gut.

FIG. 2 is a schematic diagram showing the folate metabolism.

FIG. 3 is a schematic diagram showing the system for folate receptor free (FRF) milk to generate cows and decrease rates of ASD.

FIG. 4 is a schematic diagram of the Knockdown Folate Receptor in cow fibroblast system.

FIG. 5 is a schematic diagram for nuclear transfer to generate transgenic cows system.

FIG. 6 is a schematic diagram showing the Cohort expansion and milk analyses system.

FIG. 7 is a table listing shRNA's in the library to be tested.

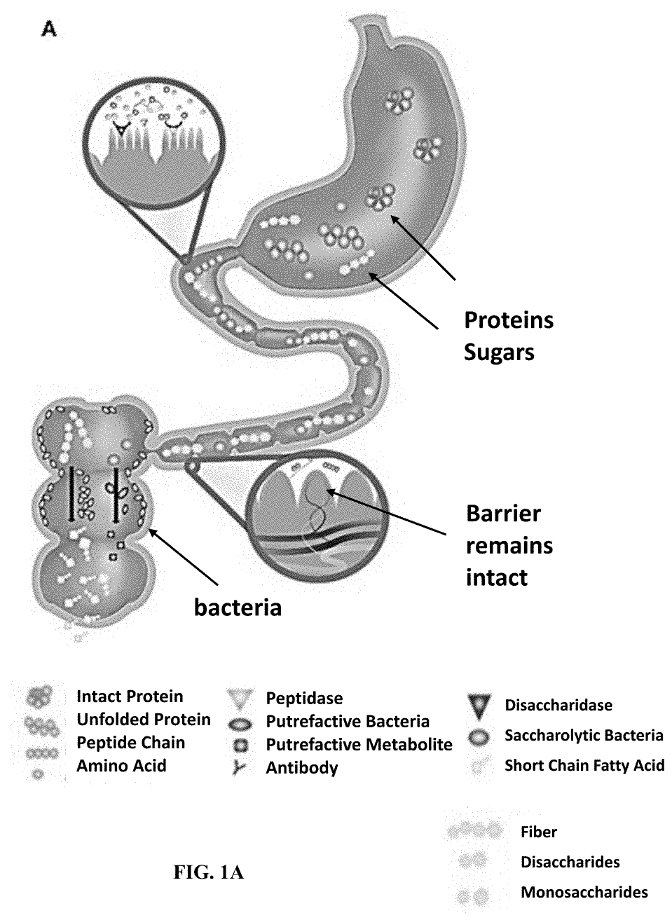

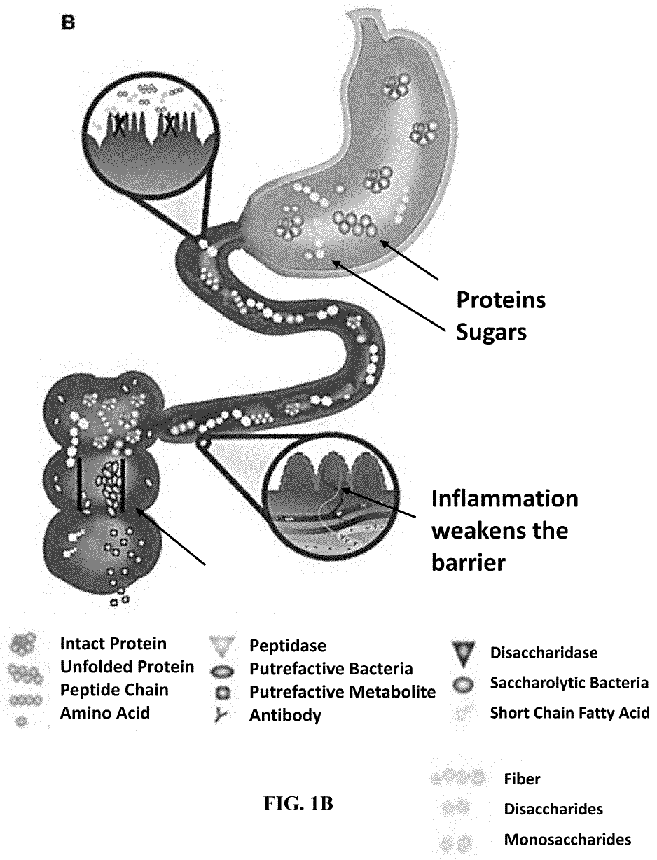

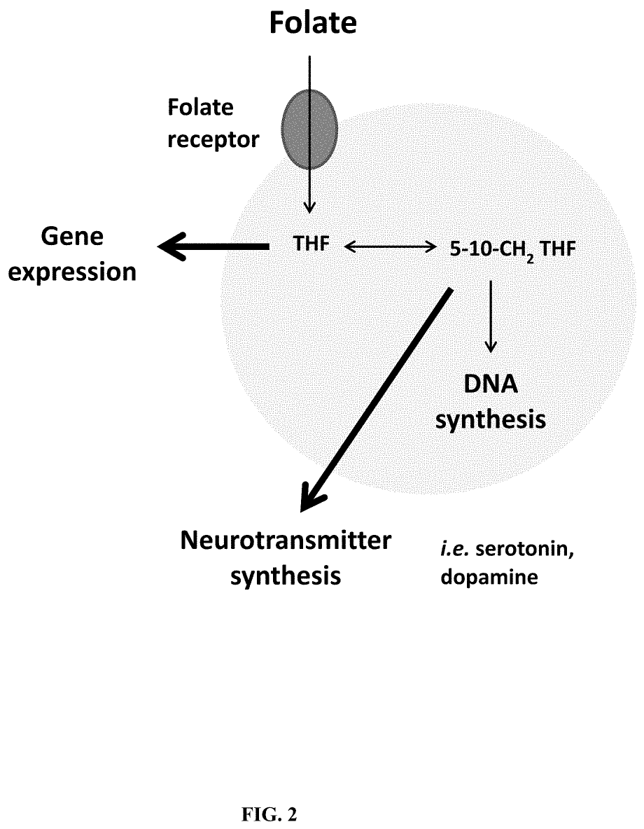

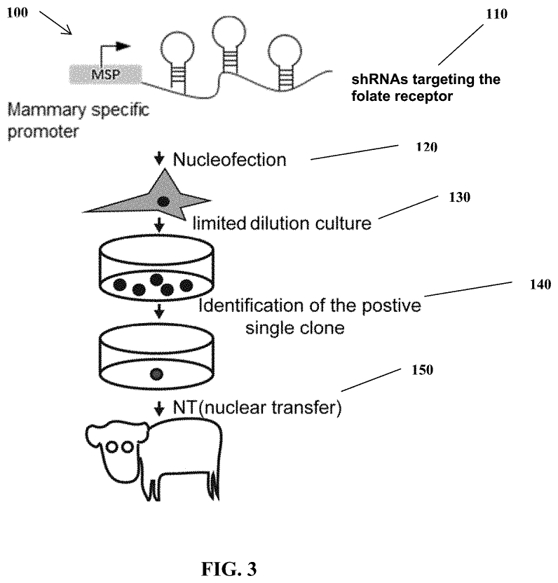

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein. The words proximal and distal are applied herein to denote specific ends of components of the instrument described herein. A proximal end refers to the end of an instrument nearer to an operator of the instrument when the instrument is being used. A distal end refers to the end of a component further from the operator and extending towards the surgical area of a patient and/or the implant.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

On embodiment are assays designed to remove folate receptor from an existing milk or milk product. The Folate Receptor Free (“FRF”) milk can further be used to create products processed from FRF milk that form part of this invention, which are derived from a source of bulk milk (i.e. milk from more than one animal) and include, but are not limited to: bulk milk; bulk milk used to make cheese whether or not the milk has been pasteurised, sterilised or otherwise treated to reduce the population of microbes prior to cheese making; milk powders, milk solids; caseins, caseinates, and casein hydrolysates; pasteurised, sterilised, preserved milks including microfiltered milks, UHT milks; low fat milks; modified or enhanced milks; ice-cream or other frozen dairy based confections; fermented milk products such as yoghurt or quark; cheeses including full fat, partial de-fatted and fat-free processed cheeses; milk whey; food products enriched through the addition of milk products such as soups; milk from which potentially allergenic molecules have been removed; confections such as chocolate; carbonated milk products, including those with added phosphate and/or citrate; infant formulations which may contain full, partially de-fatted or nonfat milk together with a number of additional supplements; liquid or powdered drink mixtures, and buttermilk and buttermilk powder.

In this aspect of the invention are assays designed to remove FR from any milk product. The methods include, but not limited to, Size exclusion chromatography, Glycophosphate inositol (GPI) capture column affinity chromatography, Folate Receptor binding antibody affinity column chromatography, Specific digestion of the folate receptor using proteases attached to the FR binding antibody, Fractionation of the milk products to remove the FR, and Affinity chromatography using ligands that specifically bind the folate receptor in milk.

Size exclusion chromatography or Fractionation of milk proteins: The 38 kDa size of the FR protein can allow for ultrafiltration of the milk either through a membrane or multiple membranes that either selectively exclude the 38 kDa FR protein or include the 38 kDa FR protein, thus depleting the milk of the FR protein. This same principle can be applied to use a size exclusion and size inclusion columns such as Sephadex 100 or Sephacryl S-200 to deplete the milk of FR.

Folate Receptor binding antibody affinity column chromatography: An antibody (polyclonal or monoclonal) generated to either bovine, goat or other animal folate receptor can be immobilized on an inert solid matrix such as agarose or Sepharose and mixed with the milk to capture the FR antigen from milk, thus selectively removing the FR antigen from milk. A goat polyclonal antibody to the bovine FOLR1/Folate Receptor Alpha from LSBio may be conjugated to the column chromatography.

The terms “folate receptor”, “FR”, or “FOLR1”, as used herein, refers to any native bovine or goat FOLR1, unless otherwise indicated. The term “FR” encompasses “full-length,” unprocessed FR as well as any form of FR that results from processing within the cell. The term also encompasses naturally occurring variants of FR, e.g., splice variants, allelic variants and isoforms. The FR proteins described herein can be isolated from a variety of sources, such as from human tissue types or from another source, or prepared by recombinant or synthetic methods. Antibodies targeting the protein sequence and Blast Identification number for the Folate receptor from cow, rat, goat and human are below:

Human Folate receptor aloha-Homo sapiens, 257 aa.
Transcript ID: FOLR1-203 ENST00000393679.5
SEQ ID NO: 1
MAQRMTTQLLLLLVWVAVVGEAQTRIAWARTELLNVCMNAKHHKEKPGPEDKLHEQCRPW
RKNACCSTNTSQEAHKDVSYLYRFNWNHCGEMAPACKRHFIQDTCLYECSPNLGPWIQQV
DQSWRKERVLNVPLCKEDCEQWWEDCRTSYTCKSNWHKGWNWTSGFNKCAVGAACQPFHF
YFPTPTVLCNEIWTHSYKVSNYSRGSGRCIQMWFDPAQGNPNEEVARFYAAAMSGAGPWA
AWPFLLSLALMLLWLLS
Rat Folate receptor aloha-Rattus norvegicus, 255 aa.
Transcript ID: Folr1-202 ENSRNOT00000079675.1
SEQ ID NO: 2
MAHLMAGQWLLLLMWMAECAQSRATRARTELLNVCMDAKHHKEKPGPEDKLHDQCSPWKT
NACCSTNTSQEAHKDISYLYRFNWNHCGTMTPECKRHFIQDTCLYECSPNLGPWIQQVDQ
SWRKERILDVPLCKEDCVLWWEDCKSSFTCKSNWHKGWNWTSGHNECPVGASCHPFTFYE
PTPAVLCEKIWSHSYKLSNYSRGSGRCIQMWFDPAQGNPNEEVARFYAEVMSGAGLREAW
LLVCSLSLVLFWVLS
Goat folate receptor alpha-Capra hircus, 251 aa,
Transcript ID: ENSCHIT00000031557.1
SEQ ID NO: 3
MAWKITPLLCLLVCVAAVGAARPRTQLLNVCMNARYHKEKPGPEDKLHGQCSPWKNNACC
FVNTSIEAHKDISSLYRFDWDHCGKMEPACKRHFIQDICLYECSPNLGPWIQEVNQSWRK
ERILNVPLCKEDCESWWEDCRTSYTCKSNWHRGWDWTSGYNQCPVKVACHRFDFYFPTPA
ALCNEIWSHSYRASNYSRGSGRCIQMWFDPVLGNPNEEVARFYAESMNGAGLHEAWPLRF
GLLLLLLWLLS
Cow folate receptor alpha-Bos Taurus, 251 aa,
Transcript ID: FOLR1-201 ENSBTAT00000071439.1
SEQ ID NO: 4
MAWKITPLLCLLVCVAAVGAARPGTQLLNVCMNARYHKEKPGPEDKLHGQCSPWKKNACC
FVNTSIEAHKDISSLYRFDWDHCGKMEPACKRHFIQDTCLYECSPNLGPWIREVNQSWRK
ERILNVPLCKEDCQSWWEDCRTSYTCKSNWHRGWDWTSGYNQCPVKAACHRFDFYFPTPA
ALCNEIWSHSYKASNYSRGSGRCIQMWFDPILDNPNEEVARFYAESMNGAGLHEAWPLRC
GLLLLLLWLLS
NCBI BLAST
Human Folate receptor-Homo sapiens, Protein sequence-257 aa,
Accession: AAB05827
SEQ ID NO: 5
maqrmttqll lllvwvavvg eaqtriawar tellnvcmna khhkekpgpe dklheqcrpw
rknaccstnt sqeahkdvsy lyrfnwnhcg emapackrhf iqdtclyecs pnlgpwiqqv
dqswrkervl nvplckedce qwwedcrtsy tcksnwhkgw nwtsgfnkca vgaacqpfhf
yfptptvlcn eiwthsykvs nysrgsgrci qmwfdpaqgn pneevarfya aamsgagpwa
awpfllslal mllwlls
Rat Folr1 protein, partial-Rattus norvegicus, Protein sequence-203 aa
ACCESSION AAI57812
SEQ ID NO: 6
mahlmagqwl lllmwmaeca qsratrarte llnvcmdakh hkekpgpedk lhdqcspwkt
naccstntsq eahkdisyly rfnwnhcgtm tpeckrhfiq dtclyecspn lgpwiqqvdq
swrkerildv plckedcvlw wedckssftc ksnwhkgwnw tsghnecpvg aschpftfyf
ptpavlceki wshsyklsny srg
Goat folate receptor 1, partial-Capra hircus, Protein sequence-63 aa,
ACCESSION AGA95980
SEQ ID NO: 7
cspwknnacc fvntsieahk disslyrfdw dhcgkmepac krhfiqdicl yecspnlgpw
iqe

Antibodies targeting the protein sequence of the goat FR include those targeting the SEQ ID NO: 8: mpwkltplll flgwmtsvcn artrtdllnv cmdakhhkae pgpedklhnq ctpwkknacc sarvsqelhk dtsslynftw dhcgkmepac qrhfiqdncl yecspnlgpw iqevnqkwrk erflnvplck edcqswwedc rtshtcksnw hrgwdwtsgs nkcpngttcr tfeayfptpa alceglwshs yklsnysrgs grciqmwfdp algnpneeva rfyasaltae awpqgirpls lclalmlslw lhd

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, bovine antibodies, goat antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “an antibody that binds to FR” refers to an antibody that is capable of binding FR with sufficient affinity such that the antibody is useful as a filtering or binding agent in targeting FR. The extent of binding of an anti-FR antibody to an unrelated, non-FR protein is less than about 10% of the binding of the antibody to FR as measured, e.g., by a radioimmunoassay (RIA). In certain embodiments, an antibody that binds to FR has a dissociation constant (Kd) of ≤1 μM, ≤100 nM, ≤10 nM, ≤1 nM, or ≤0.1 nM.

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

Specific digestion of the folate receptor using proteases attached to the FR binding antibody. It may also be possible to attach a protease such as trypsin or carboxypeptidase to a FR specific antibody and use it to selectively digest the FR protein, thus rendering it less antigenic.

As used herein, the terms “protease” and “proteinase” refer to an enzyme protein that has the ability to break down other proteins. A protease has the ability to conduct “proteolysis,” which begins protein catabolism by hydrolysis of peptide bonds that link amino acids together in a peptide or polypeptide chain forming the protein. This activity of a protease as a protein-digesting enzyme is referred to as “proteolytic activity.” Many well-known procedures exist for measuring proteolytic activity (See e.g., Kalisz, “Microbial Proteinases,” In: Fiechter (ed.), Advances in Biochemical Engineering/Biotechnology, (1988)). For example, proteolytic activity may be ascertained by comparative assays which analyze the respective protease's ability to hydrolyze a commercial substrate. Exemplary substrates useful in the analysis of protease or proteolytic activity, include, but are not limited to, di-methyl casein (Sigma C-9801), bovine collagen (Sigma C-9879), bovine elastin (Sigma E-1625), and bovine keratin (ICN Biomedical 902111). Colorimetric assays utilizing these substrates are well known in the art (See e.g., WO 99/34011 and U.S. Pat. No. 6,376,450, both of which are incorporated herein by reference). The pNA assay (See e.g., Del Mar et al., Anal. Biochem. 99:316-320 [1979]) also finds use in determining the active enzyme concentration for fractions collected during gradient elution. This assay measures the rate at which p-nitroaniline is released as the enzyme hydrolyzes the soluble synthetic substrate, succinyl-alanine-alanine-proline-phenylalanine-p-nitroanilide (suc-AAPF-pNA). The rate of production of yellow color from the hydrolysis reaction is measured at 410 nm on a spectrophotometer and is proportional to the active enzyme concentration. In addition, absorbance measurements at 280 nanometers (nm) can be used to determine the total protein concentration. The active enzyme/total protein ratio gives the enzyme purity.

Affinity chromatography using ligands that specifically bind the folate receptor in milk. Ligands such as folic acid bind to the FR with high affinity (Kd about100 pM) and therefore immobilizing the folic acid on an inert matrix such as agarose or Sepharose and mixing with milk that contains the apo receptor will allow for the receptor to bind to the folic acid and then can be removed from the milk.

“Folic Acid” as used herein means folic acid, a folic acid analog, or another folate receptor-binding molecule. Analogs of folate that can be used include folinic acid, pteropolyglutamic acid, and folate receptor-binding pteridines such as tetrahydropterins, dihydrofolates, tetrahydrofolates, and their deaza and dideaza analogs. The tenms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N10-methylfolate, 2-deamino-hydroxyfolate, deaza analogs such as 1-deazamethopterin or 3-deazamethopterin, and 3′,5′-dichloro-4-amino-4-deoxy-N 10-methylpteroylglutamic acid (dichloromethotrexate).

“High Affinity” may be about Kd=10⁻¹⁰M.

Validation of FRF milk:

1. 3H folic acid binding assay for functional FR.

Milk samples will be incubated with radioactive folic acid for 30 min followed by separation of protein -bound folic acid from free folic acid using dextran coated charcoal. The method is sensitive enough to detect picograms of bound folic acid and will provide a measure of functional folate receptor in the milk.

2. It is likely that some non-functional FR antigen may be present in the milk. This will be measured by a sequential ELISA based assay for the detection of FR antigen.

For this assay, sets of ELISA plates will be coated with the FR antibody. One set will be incubated with known amounts of FR antigen to generate a standard curve. Another set will be incubated with varying amounts of FRF milk. This first incubation can be for a few hours to overnight. To both sets, we will add FR antigen saturated with 3Hfolic acid and incubate for 20 min. The principle of the assay is based on less 3Hfolic acid binding to the antibody in the plate as the concentration of the antigen in the first incubation increases. The antigen in the FRF milk will also follow the same principle and allow us to determine if any nonfunctional fragment of the antigen is still present in the FRF milk.

3. Western blot of milk to detect FR antigen.

Validation of FRF Milk:

The validation of the FR free milk is below:

1.3H folic acid binding assay for functional FR.

Milk samples are incubated with radioactive folic acid for 30 min followed by separation of protein-bound folic acid from free folic acid using dextran coated charcoal. The method is sensitive enough to detect picograms of bound folic acid and provides a measure of functional folate receptor in the milk.

2. Some non-functional FR antigen may be present in the milk. This will be measured by a sequential ELISA based assay for the detection of FR antigen.

For this assay sets of ELISA plates are coated with the FR antibody. One set is incubated with known amounts of FR antigen to generate a standard curve. Another set is incubated with varying amounts of FRF milk. This first incubation can be from about a few hours to about 24 hours. To both sets, FR antigen saturated with 3Hfolic acid is added and incubated for about 20 min. The principle of the assay is based on less 3Hfolic acid binding to the antibody in the plate as the concentration of the antigen in the first incubation increases. The antigen in the FRF milk also follows the same principle to determine if any nonfunctional fragment of the antigen is still present in the FRF milk.

3. Western blot of milk to detect FR antigen.

Western blot analyses is performed using antibodies against FR. For these experiments, samples are incubated in 1 x SDS-PAGE sample loading buffer (Invitrogen) with or without 20 mM DTT, boiled for 10 min, and electrophoresed on 4-12% gradient SDS-PAGE gels. Protein was transferred to PVDF membrane and blots probed as described above. Gels are run using Benchmark™ prestained protein ladder (NOVEX®). Gels using FR proteins were also visualized via silverstaining to ensure standard against FR.

In other embodiments, the level of FR is determined by radioimmunoassay, immunofluorimetry, immunoprecipitation, equilibrium dialysis, immunodiffusion, electrochemiluminescence (ECL) immunoassay, immunohistochemistry, fluorescence-activated cell sorting (FACS) or other ELISA assays.

In another embodiment are methods to engineer cows and goats that produce FRF milk. Using genetic engineering methods to introduce RNAi triggers to specifically silence expression of the bovine folate receptor in the mammary gland, a bovine FRF milk product can be obtained by milking the progeny of these genetically modified cows. This embodiment includes an RNAi method comprising: identifying a plurality of shRNAs that target the bovine folate receptor; creating a plurality of mammary specific shRNA-expressing constructs; inserting the plurality of mammary specific shRNA-expressing constructs into a plurality of cow fibroblasts using a transduction method; using somatic cell transfer of fibroblasts to generate a cow; and validating that the milk produced from the cow is deplete of the folate receptor. Another embodiment is an RNAi method comprising: identifying a plurality of shRNAs that target the bovine folate receptor; creating a plurality of shRNA-expressing constructs; using CRISPR to insert the plurality of shRNA-expressing constructs into a mammary specific gene in cow fibroblasts; using somatic cell transfer of fibroblasts to generate a cow; and validating that the milk produced from the cow is deplete of the folate receptor. Another embodiment is an RNAi method comprising: identifying a plurality of shRNAs that target the bovine folate receptor; creating a plurality of shRNA-expressing constructs; using CRISPR and pronuclear injection into cow embryos to insert plurality of shRNA-expressing constructs to generate a transgenic cow; and validating that the milk produced from the transgenic cow is deplete of the folate receptor. Another embodiment of a humanization method comprising: using CRISPR to knockout a bovine receptor and replace the bovine receptor and with a human folate receptor; targeting cow fibroblasts, and then using somatic nuclear transfer or pronuclear injecting of a plurality of CRISPR reagents into a plurality of cow embryos; and validating that the milk produced from the cow expresses human folate receptor and is not antigenic.

In another embodiment are methods to supplement FRF milk with a form of folate (these include folic acid, DL folinic acid, L-folinic acid or methylfolate) to compensate for the removal of natural folate bound to the folate receptor.

In another embodiment of the technology, live-stock devoid of folate receptor may be produced by a combination of recombinant technology and/or selective inactivation of the folate receptor gene with appropriate intervention to maintain folate metabolism and viability of the progeny from embryonic development to adulthood.

In one embodiment, the system to generate folate receptor free milk 100 by genetically engineering livestock is shown in FIG. 3 and comprises generating shRNAs targeting the folate receptor 110, nucleofection 120 of the shRNAs, limited dilution culture 130, identifying the positive single clone 140, and conducting nuclear transfer into a cow 150. Humanized cows have been produced to mirror human breast milk and is generally accepted for human consumption. While cows have been described, other mammals may be used such as murines, simians, humans, farm animals, sport animals, and pets. Tissues, cells and their progeny of a biological entity obtained in vivo or cultured in vitro are also encompassed.

The shRNA's targeting the folate receptor will be mapped against the Bos taurus folate receptor 1 (FOLR1), and the mRNA NCBI Reference Sequence: NM_001206532.1, GenBank Graphics >NM_001206532.1 Bos taurus folate receptor 1 (FOLR1), mRNA, Seq. ID NO. 9:

CCACTAAGCCCTTCTGTCCCCTAGAGTGAGGCAAGGGGGACTGCA
GAGCAGAACAGAAGCCTGAGCCAGACGAACAGCCAGCATTCCTCC
CAGGAAATTGGTTCTTCAGAGACAGGCATGGCCTGGAAGATCACA
CCACTGCTGTGCCTTTTAGTGTGTGTGGCTGCTGTGGGGGCAGCC
CGGCCCGGGACTCAGCTTCTCAACGTCTGCATGAATGCCAGGTAC
CATAAGGAAAAACCAGGTCCCGAGGACAAGTTACATGGACAGTGC
AGCCCTTGGAAAAAGAATGCCTGCTGCTTTGTCAACACCAGCATA
GAAGCCCATAAGGATATTTCCAGCCTGTACAGATTCGACTGGGAC
CACTGCGGCAAGATGGAGCCTGCATGCAAGCGCCACTTTATTCAG
GACACCTGTCTCTACGAGTGCTCACCTAATCTGGGGCCCTGGATC
CGGGAGGTGAATCAGAGCTGGCGCAAAGAACGGATCCTGAATGTG
CCCCTCTGCAAAGAGGACTGTCAGAGCTGGTGGGAAGACTGCCGC
ACCTCCTACACCTGCAAGAGCAACTGGCACAGGGGCTGGGACTGG
ACCTCAGGGTACAACCAGTGCCCAGTGAAAGCTGCCTGCCACCGC
TTCGACTTCTACTTCCCGACGCCTGCTGCTCTGTGCAATGAAATC
TGGAGTCACTCCTACAAAGCCAGCAACTACAGTCGTGGCAGCGGC
CGCTGCATTCAGATGTGGTTCGACCCCATCCTGGACAACCCCAAC
GAGGAGGTGGCGAGATTCTATGCTGAGAGCATGAATGGGGCTGGG
CTCCATGAGGCCTGGCCTCTGCGTTGCGGCCTGCTCTTATTGTTA
CTCTGGCTGCTCAGTTGAGCTCCTATTATTTTTTGACACCTGGAA
ATCCCTGCCCCGTTAAGCTCCACAGTCAGTTTGTTCCTTGGTGGA
AACTGTTTGAATAAAAAGTCAATCACCCTAAAAAAAAAAAAAAAA

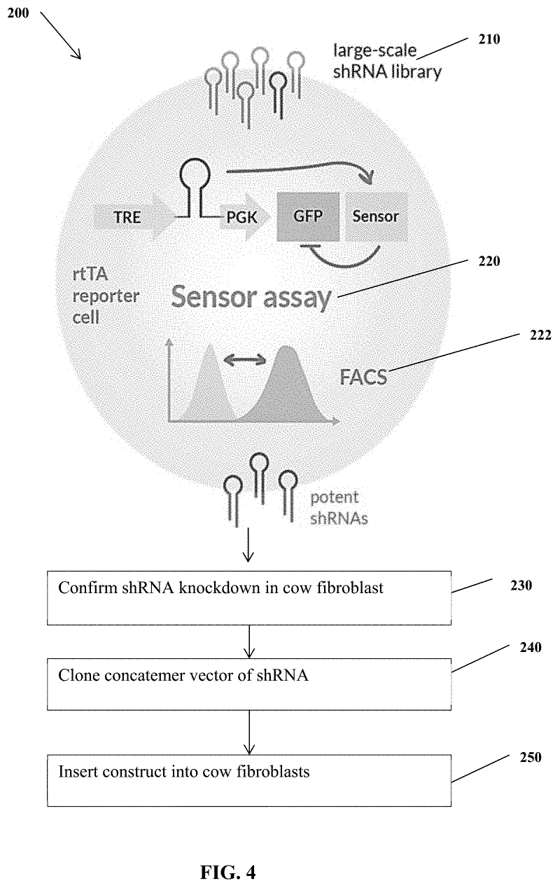

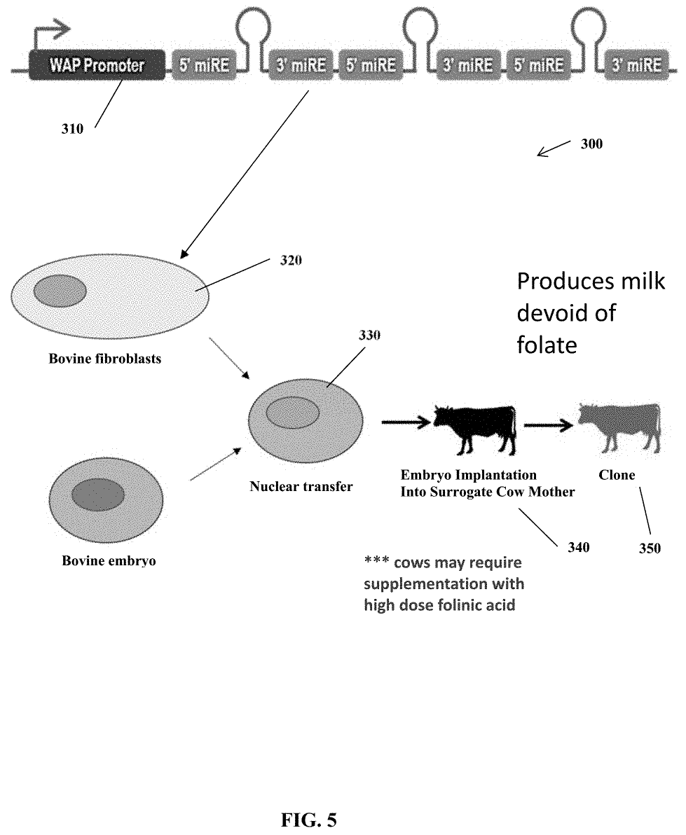

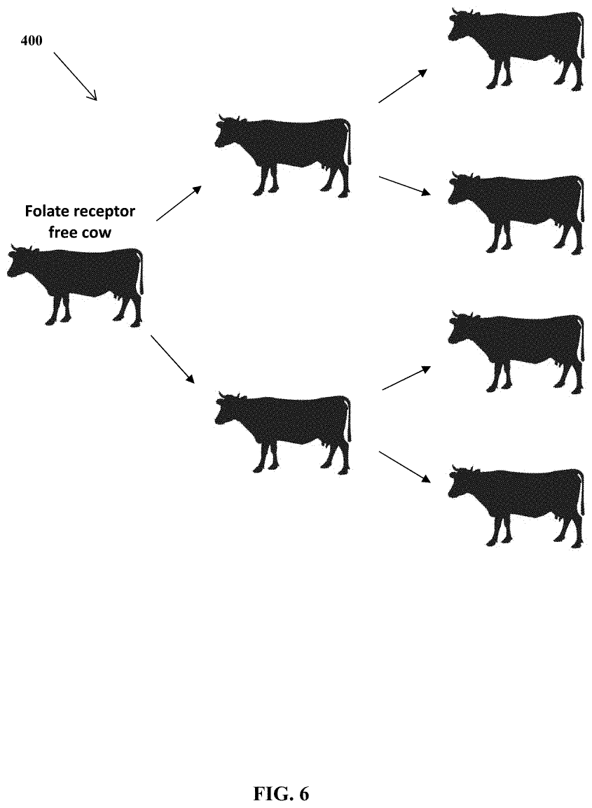

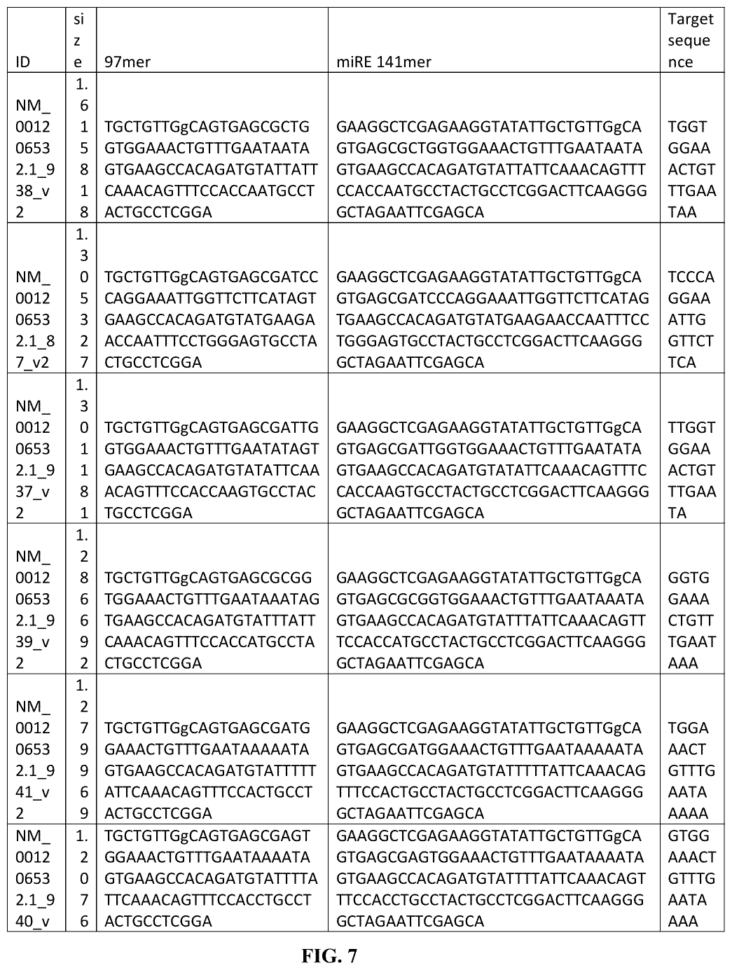

In one embodiment, a Knockdown Folate Receptor in cow fibroblast system 200 is shown in FIG. 4. The first step is to identify shRNA's in a large scale shRNA library 210 and then validate the shRNA's via sensor assay 210, which is to find shRNAs that potently and specifically silence the folate receptor. The third step is to confirm shRNA knockdown in cow fibroblast 230. The forth step is to clone a concatemer vector of shRNA 240. In one embodiment, the construct is a mammary specific promoter-shRNA-shRNA-shRNA. The final step is to insert the concatemer vector construct into cow fibroblasts 240.

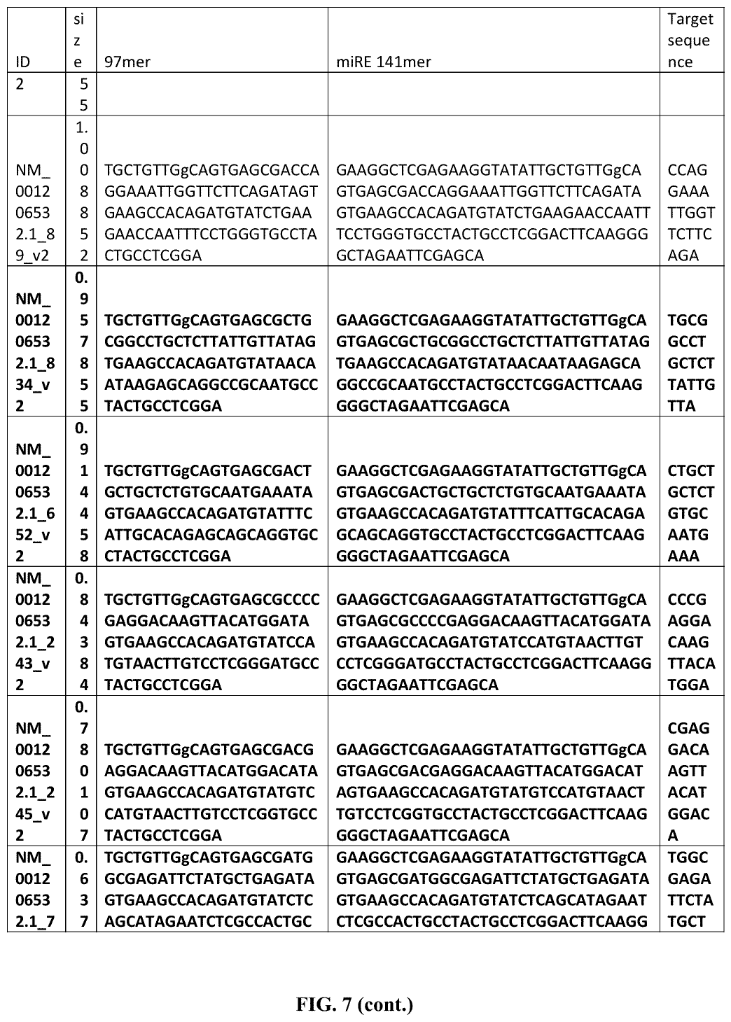

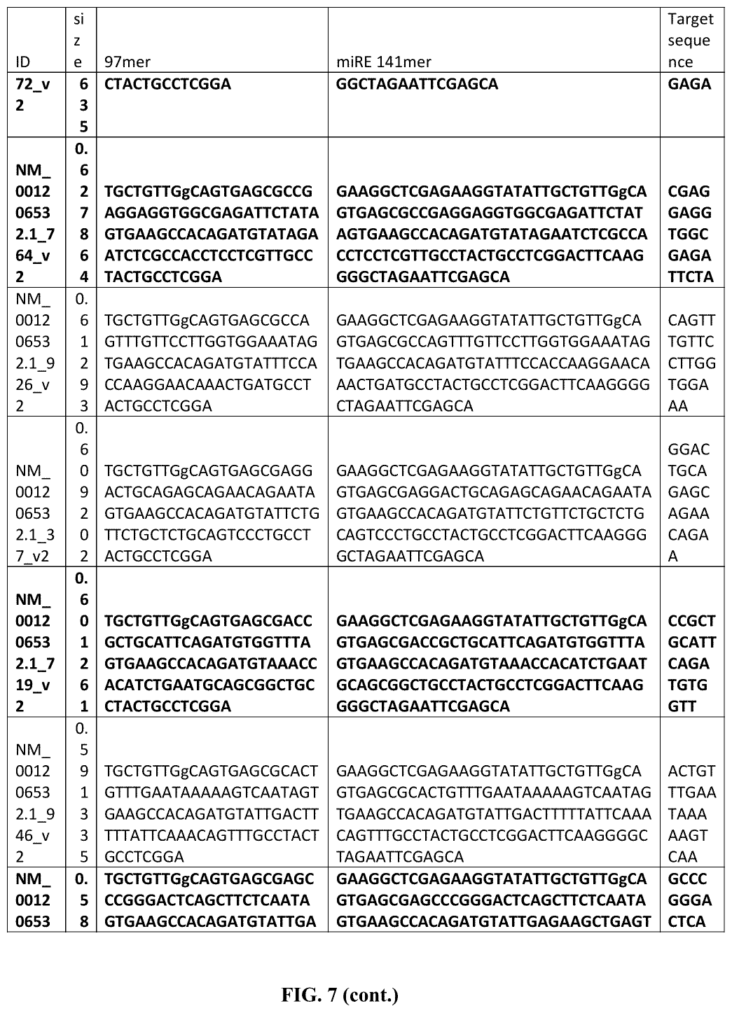

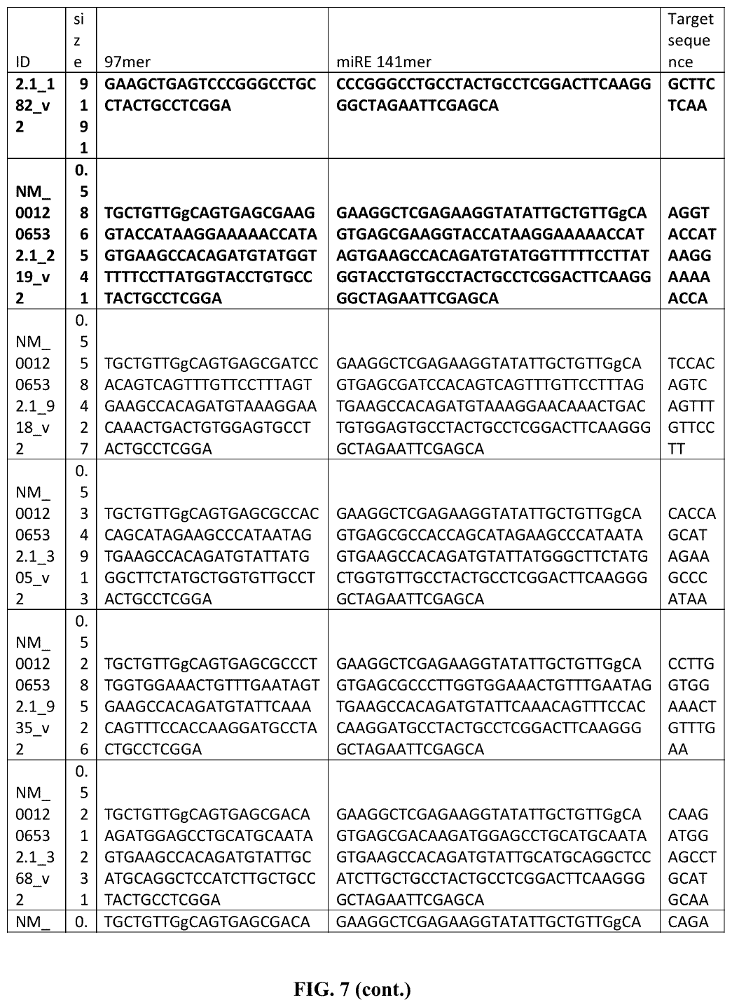

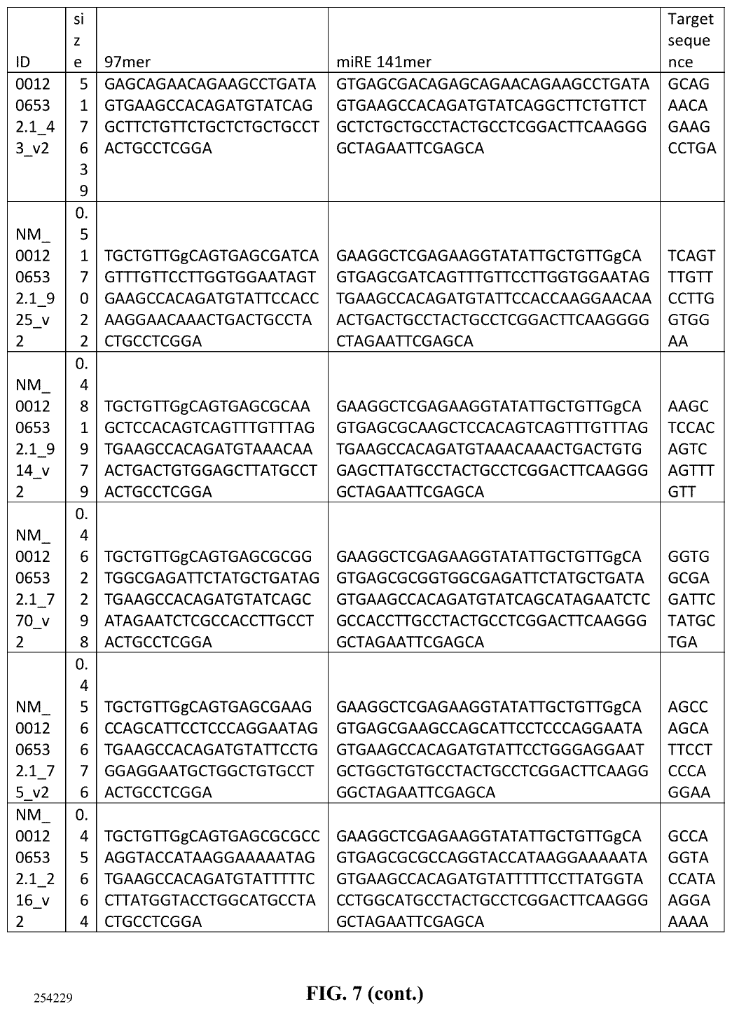

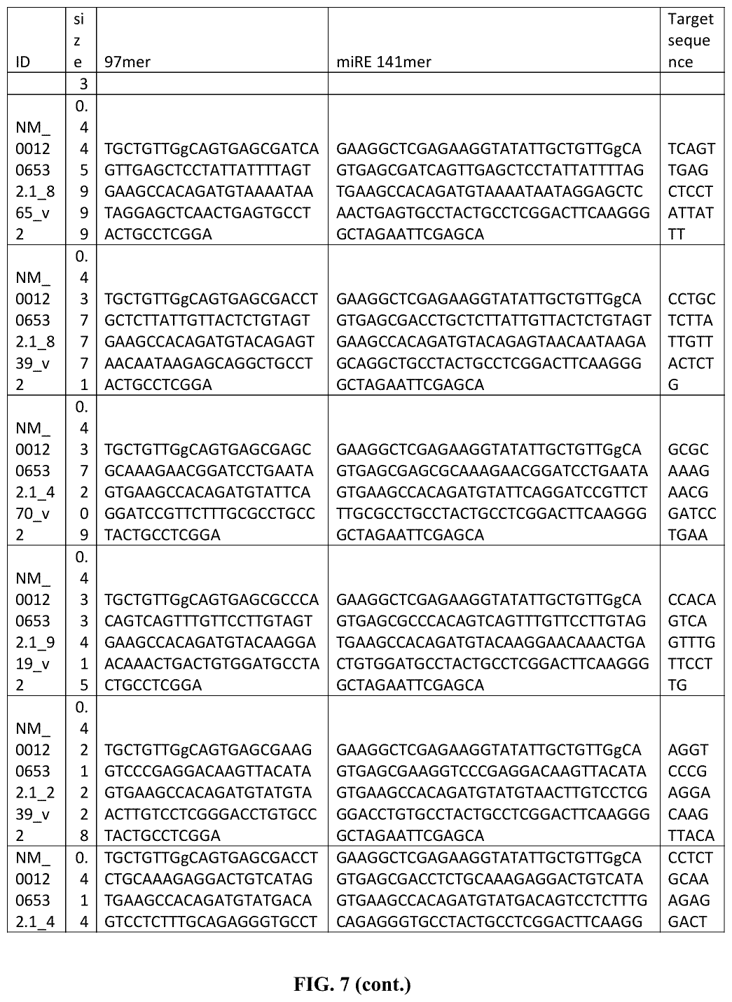

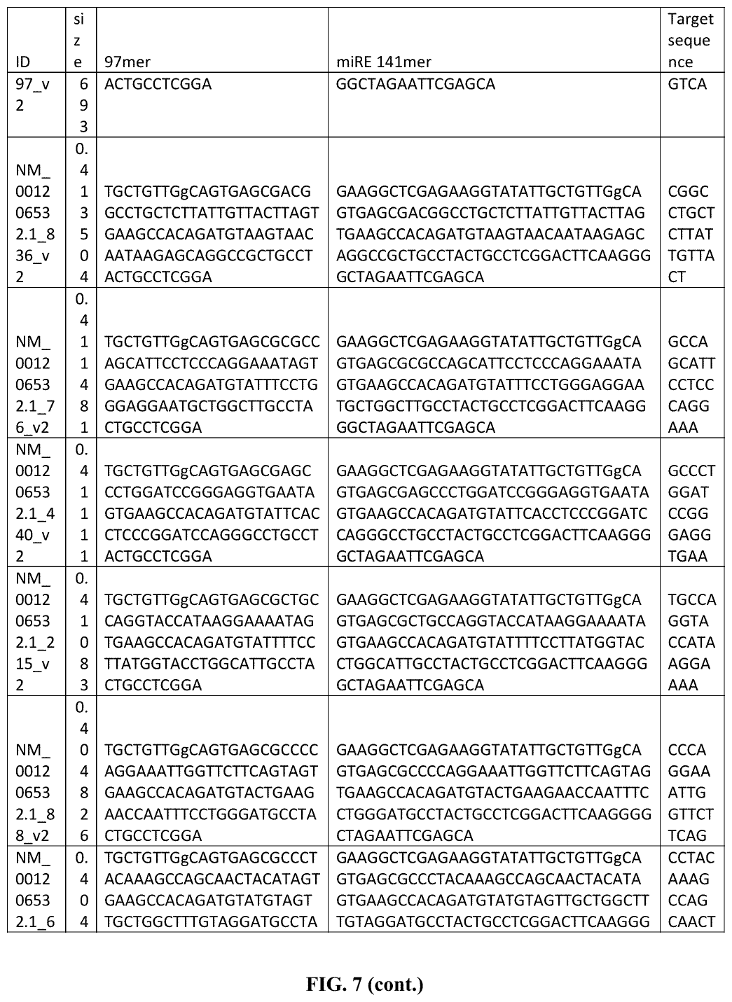

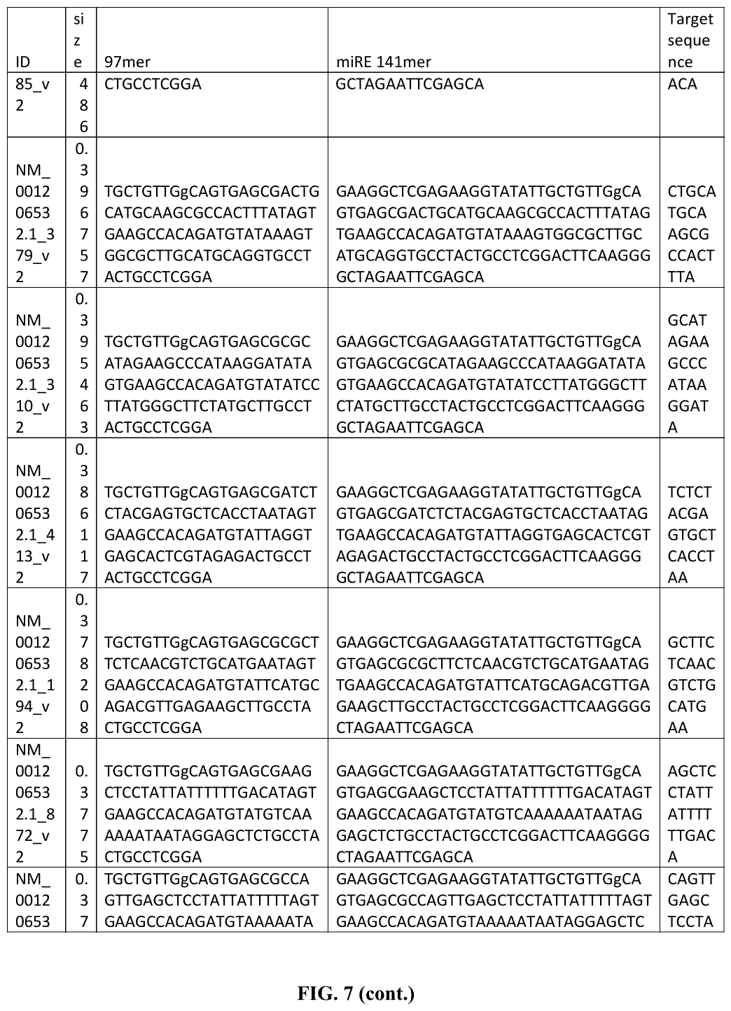

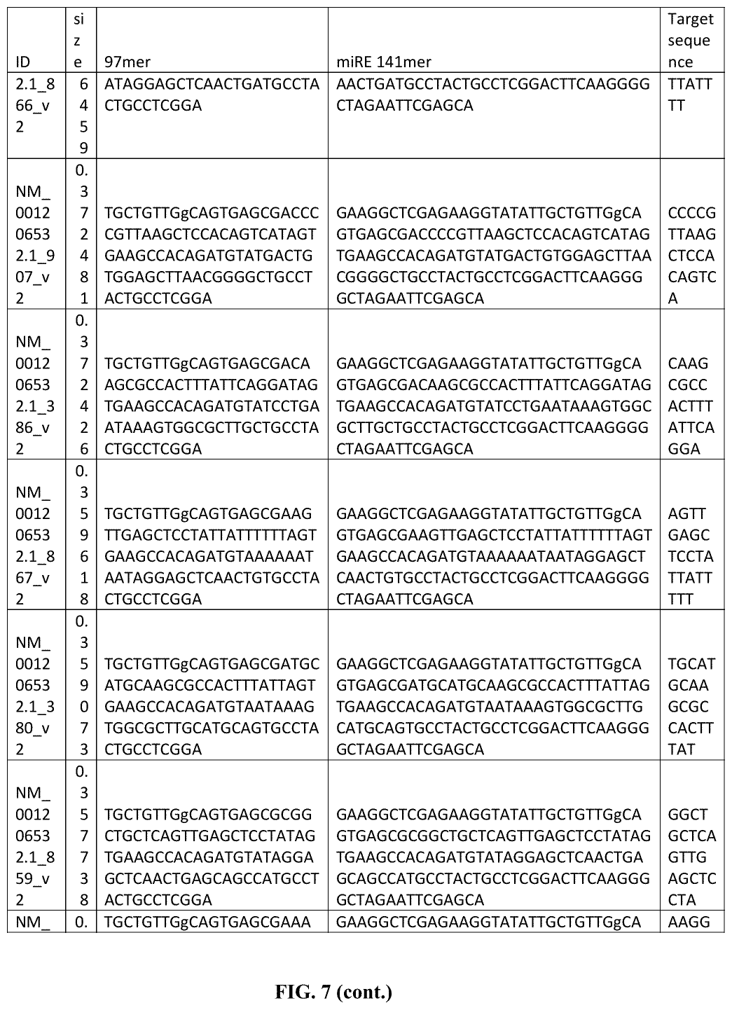

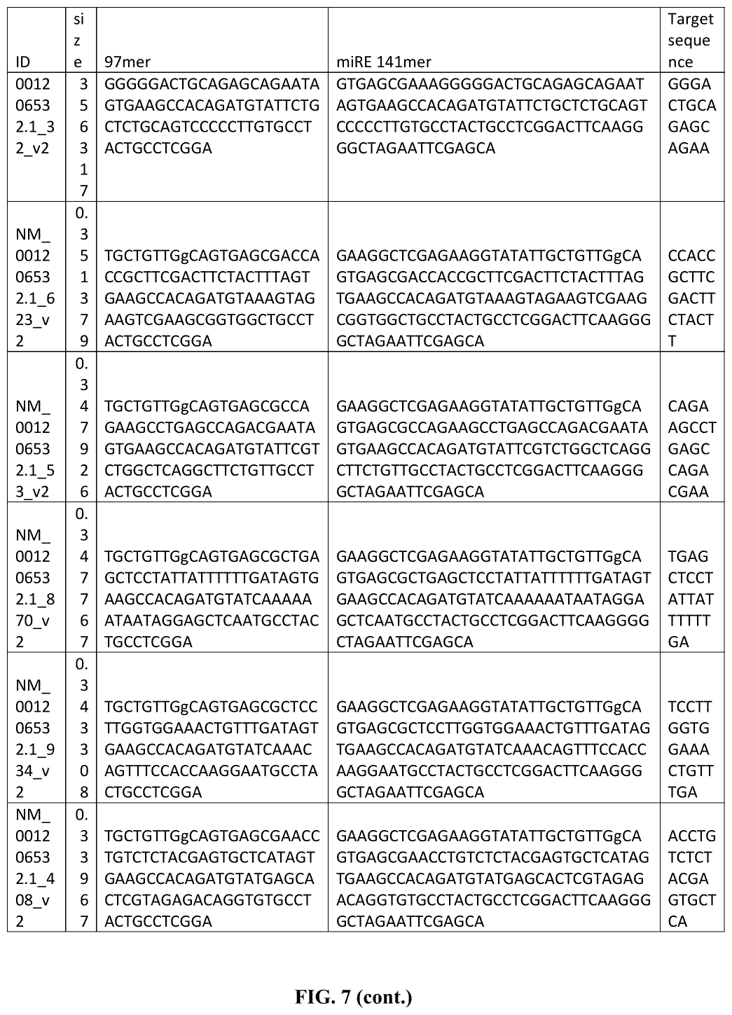

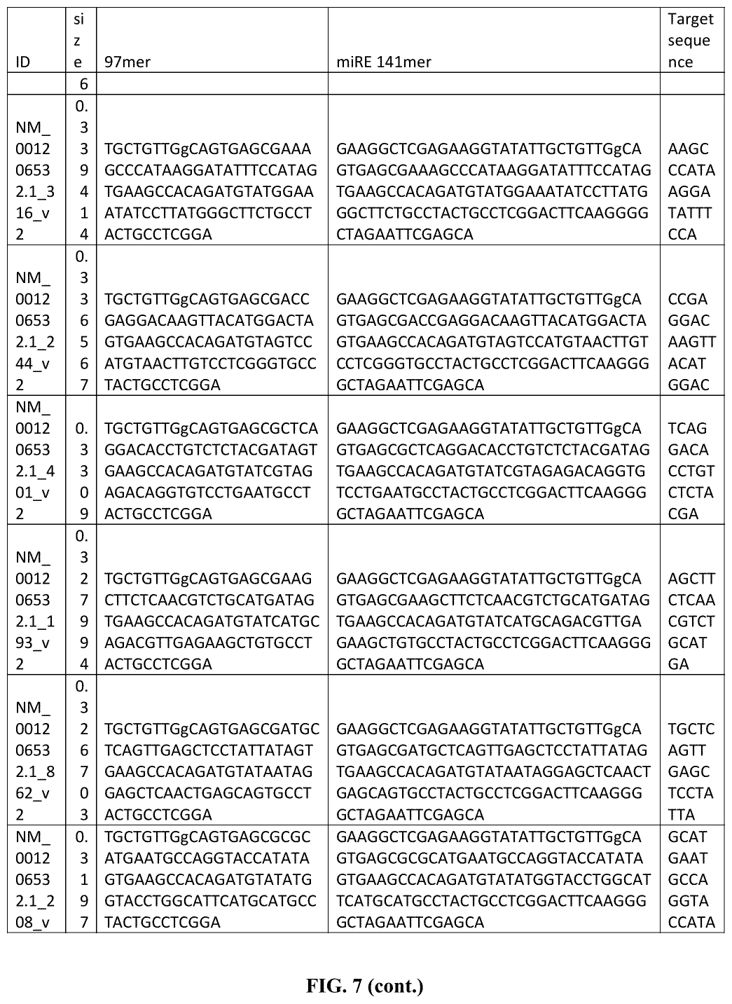

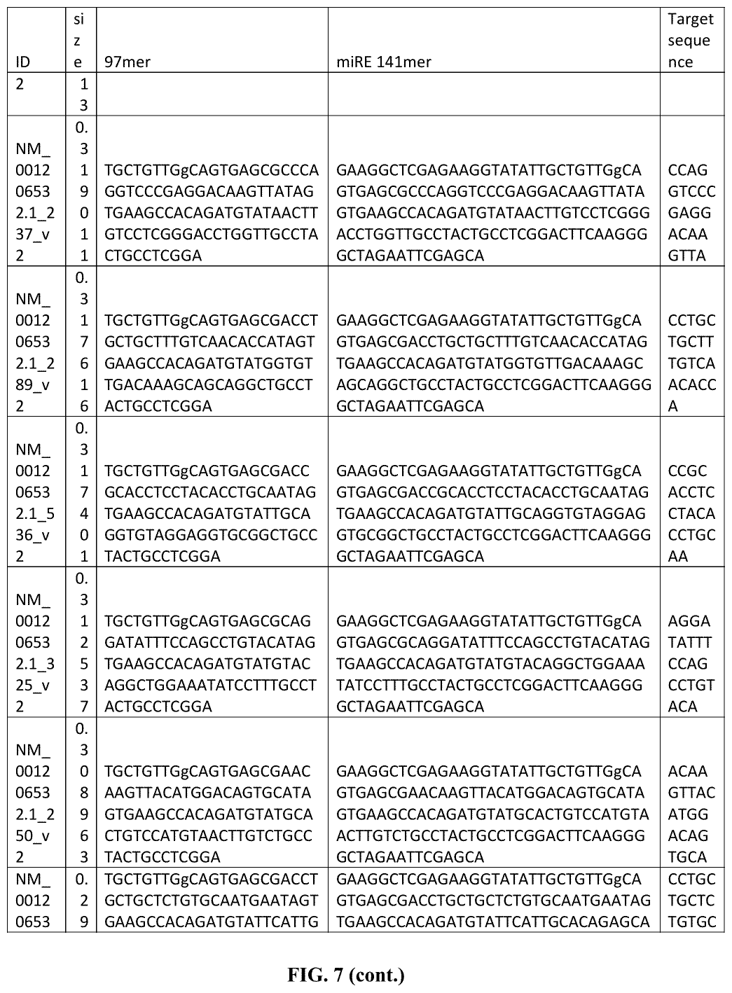

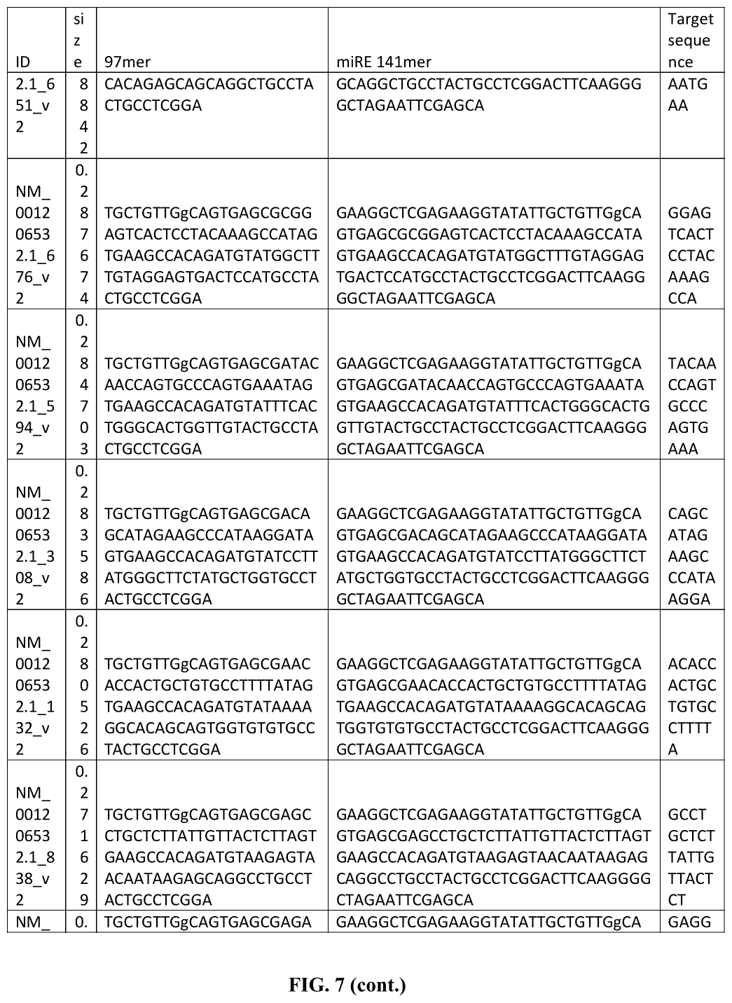

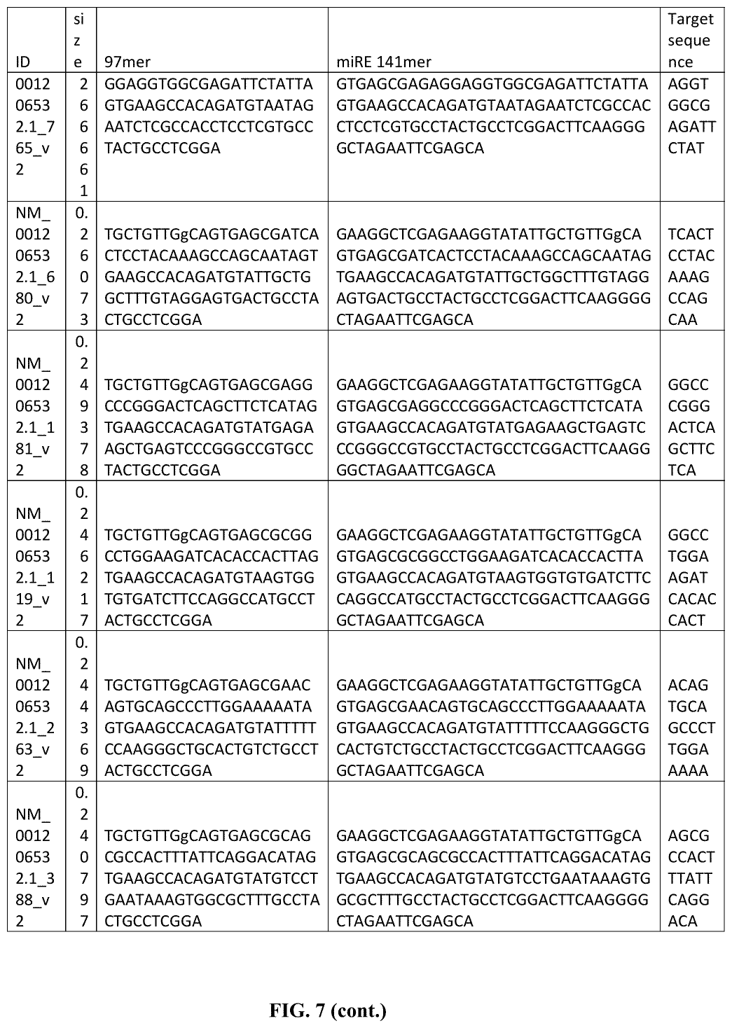

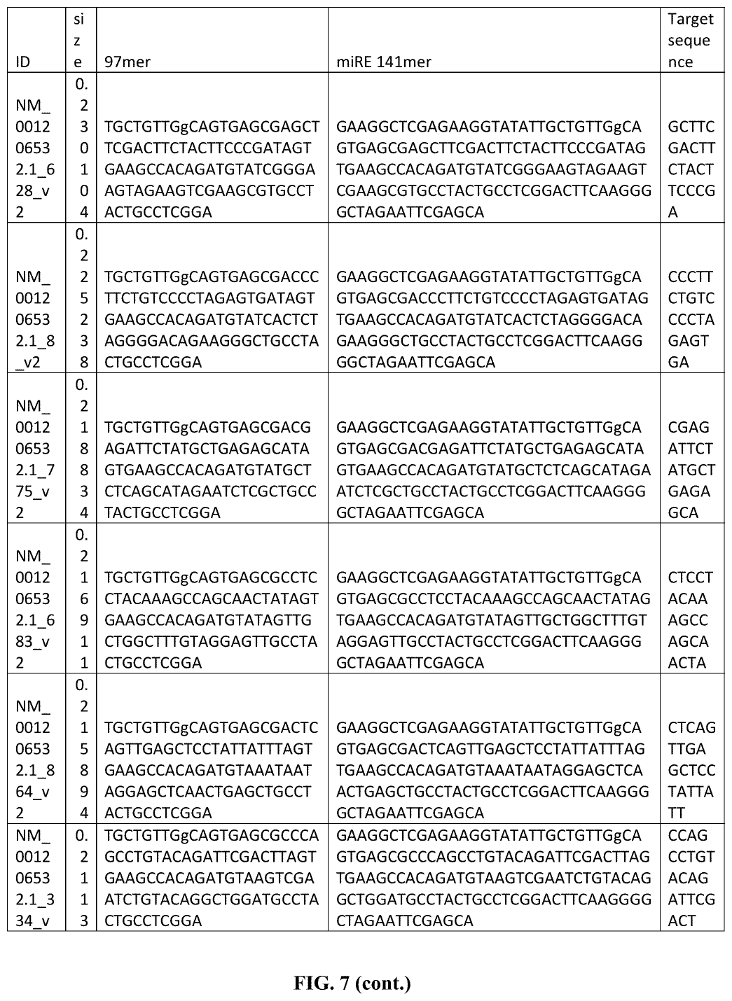

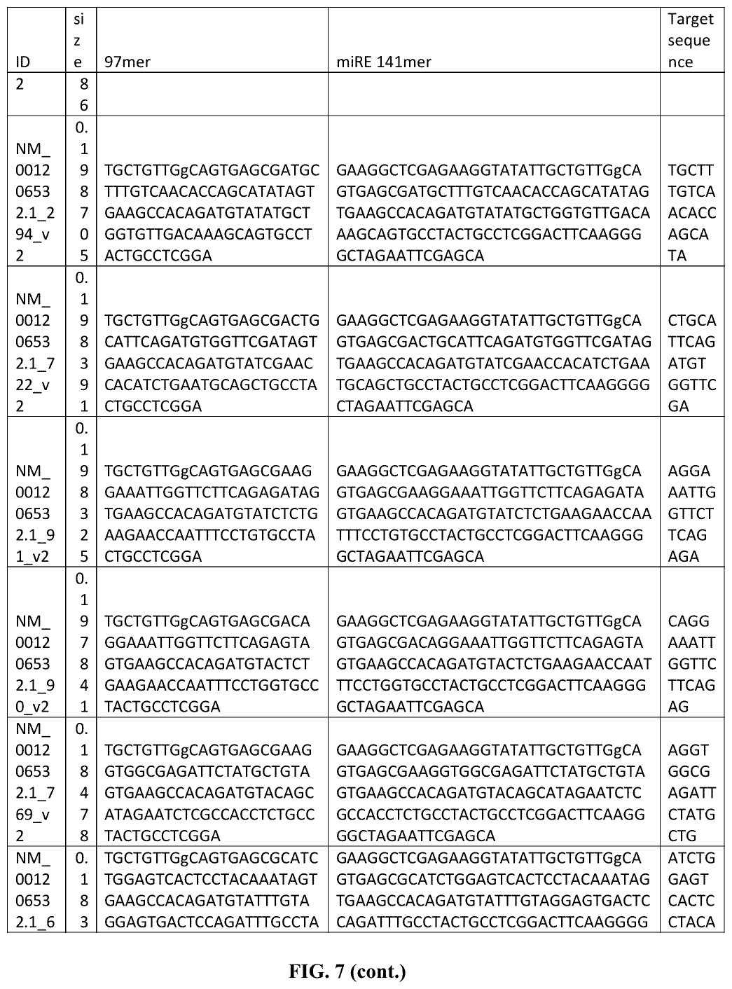

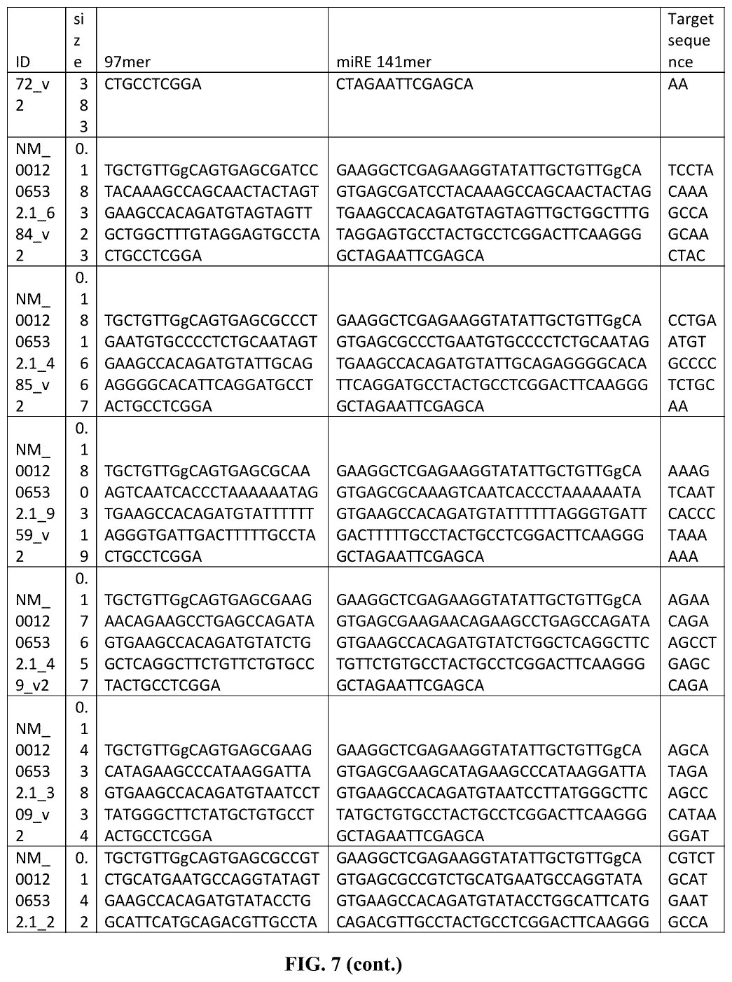

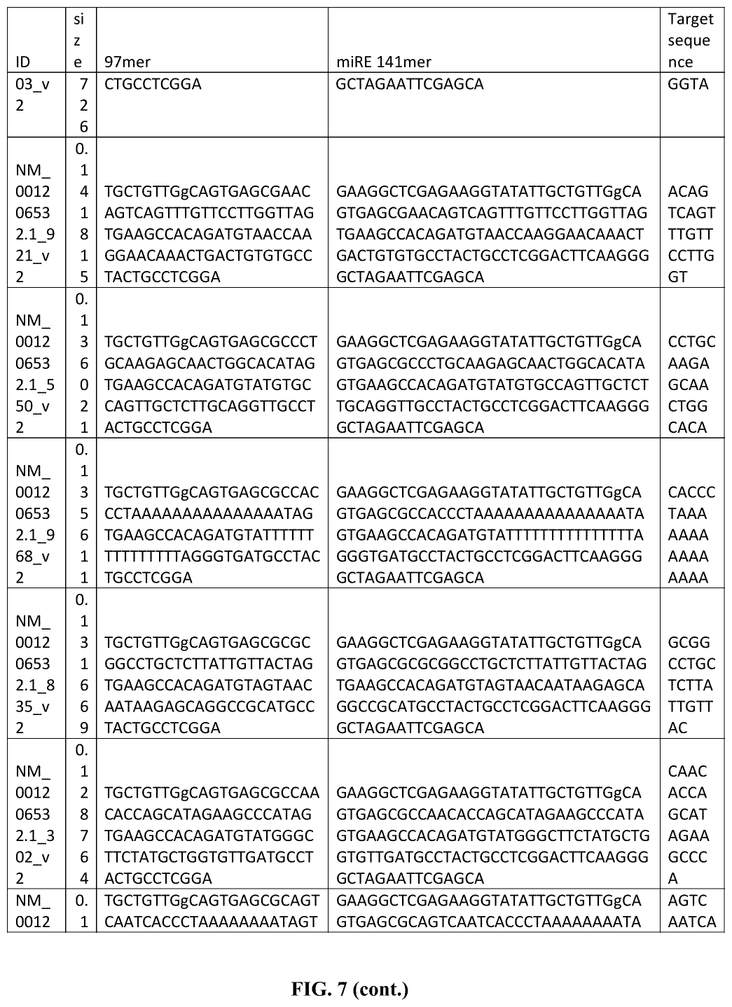

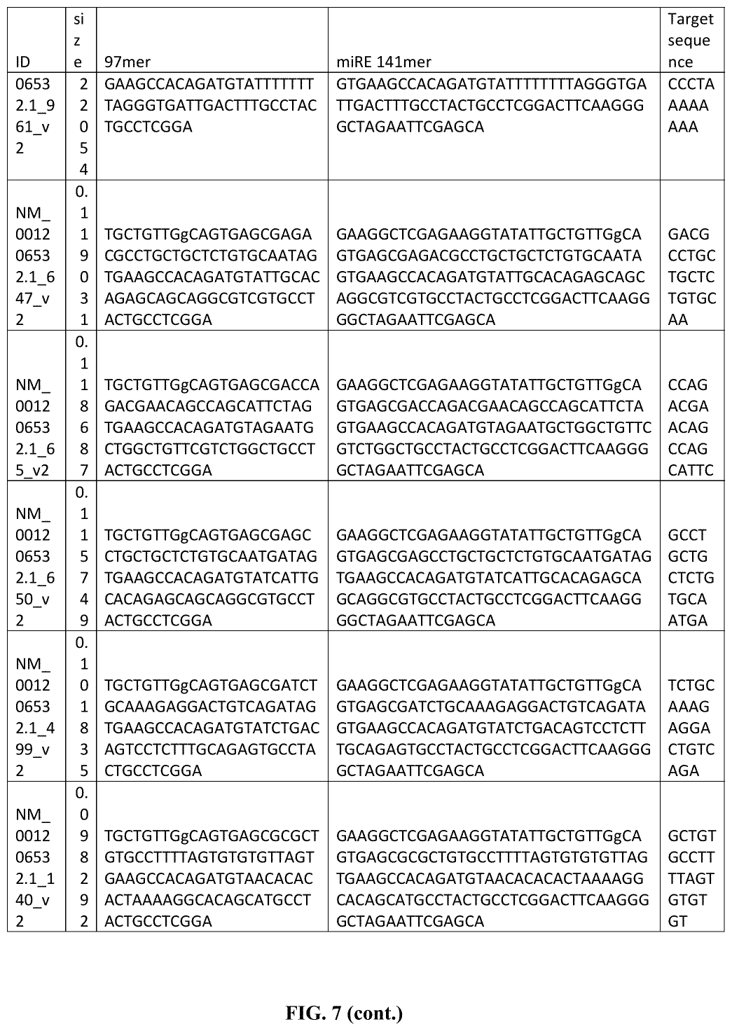

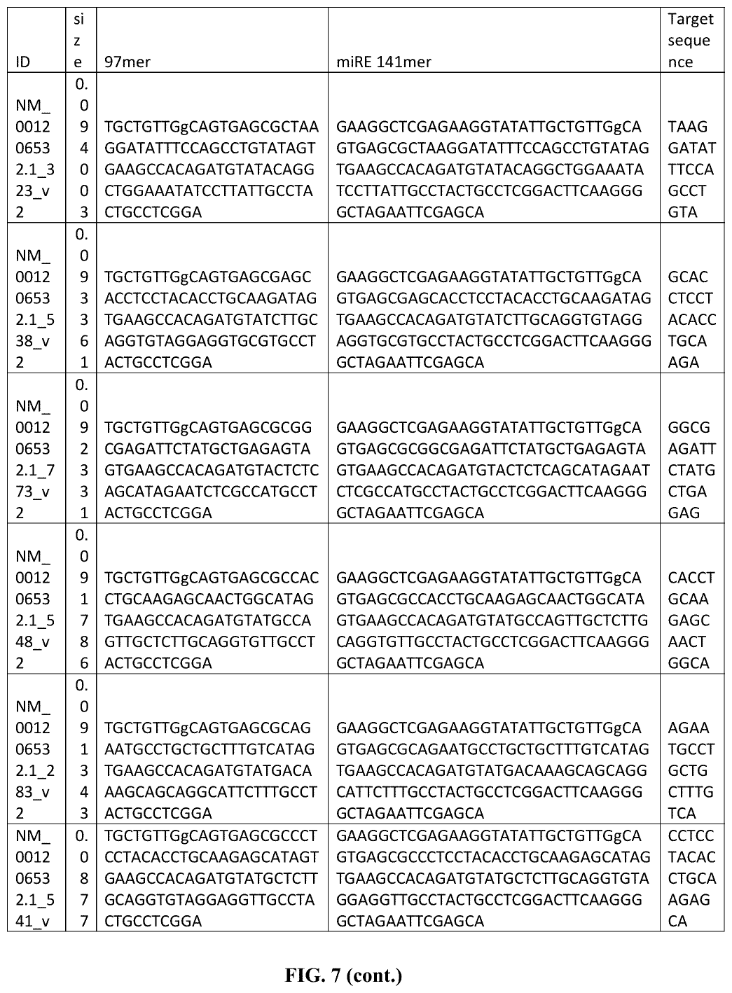

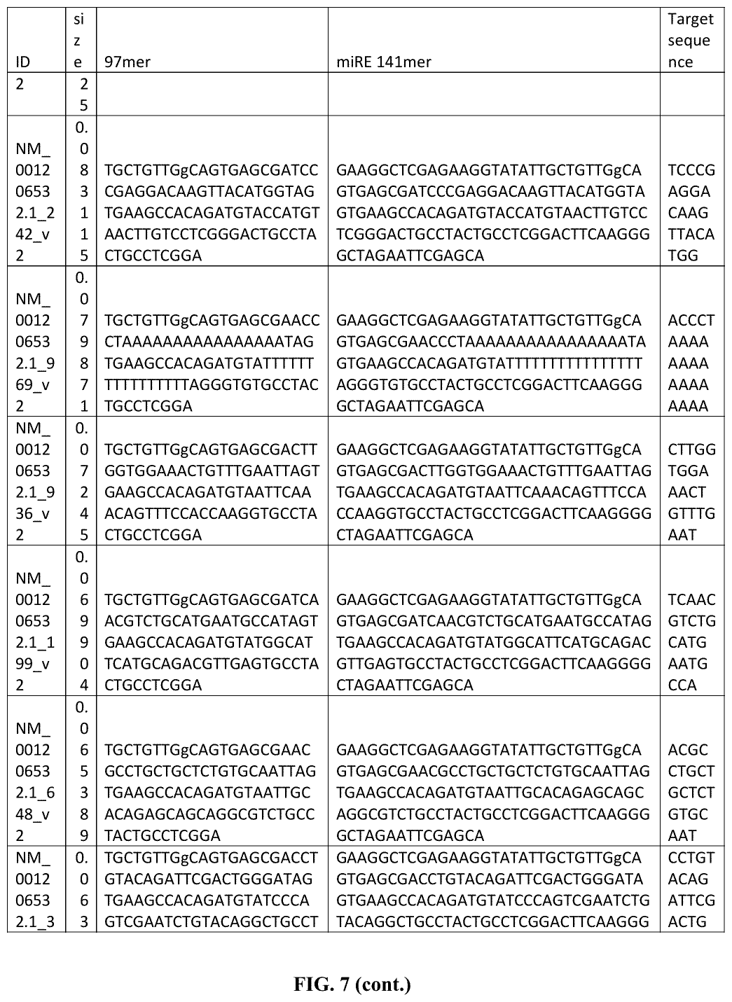

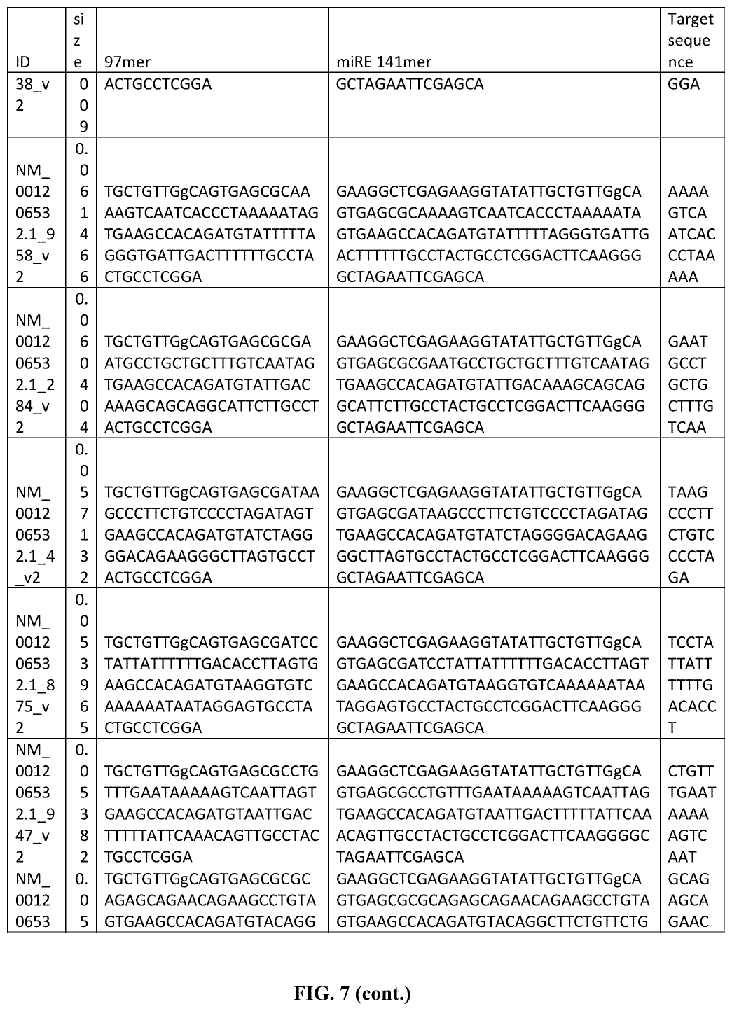

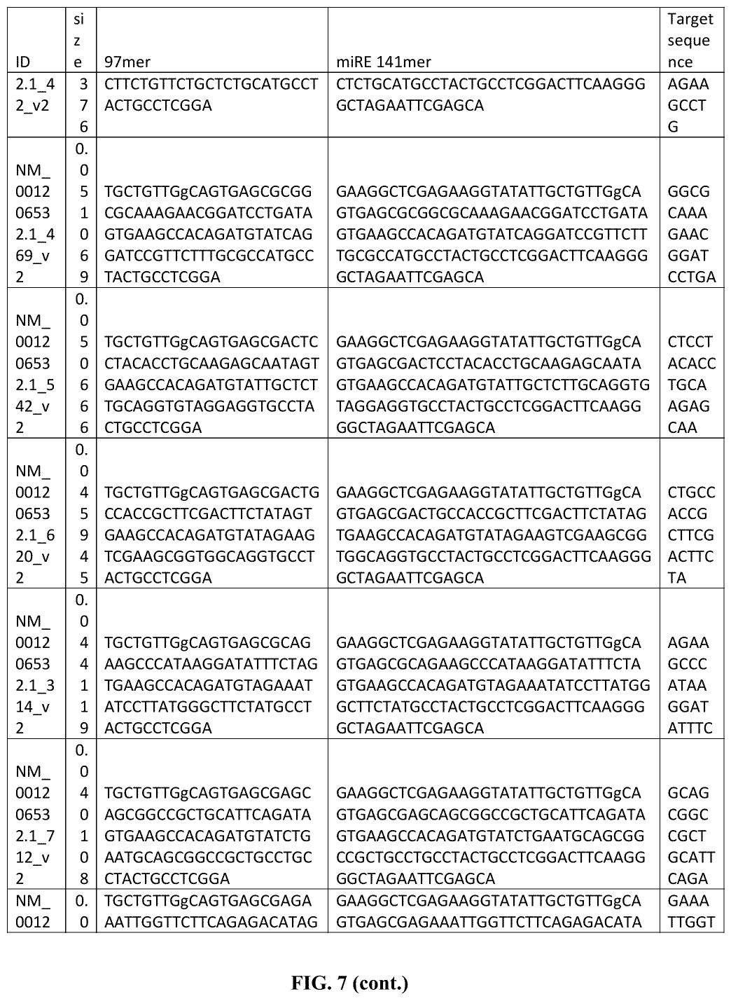

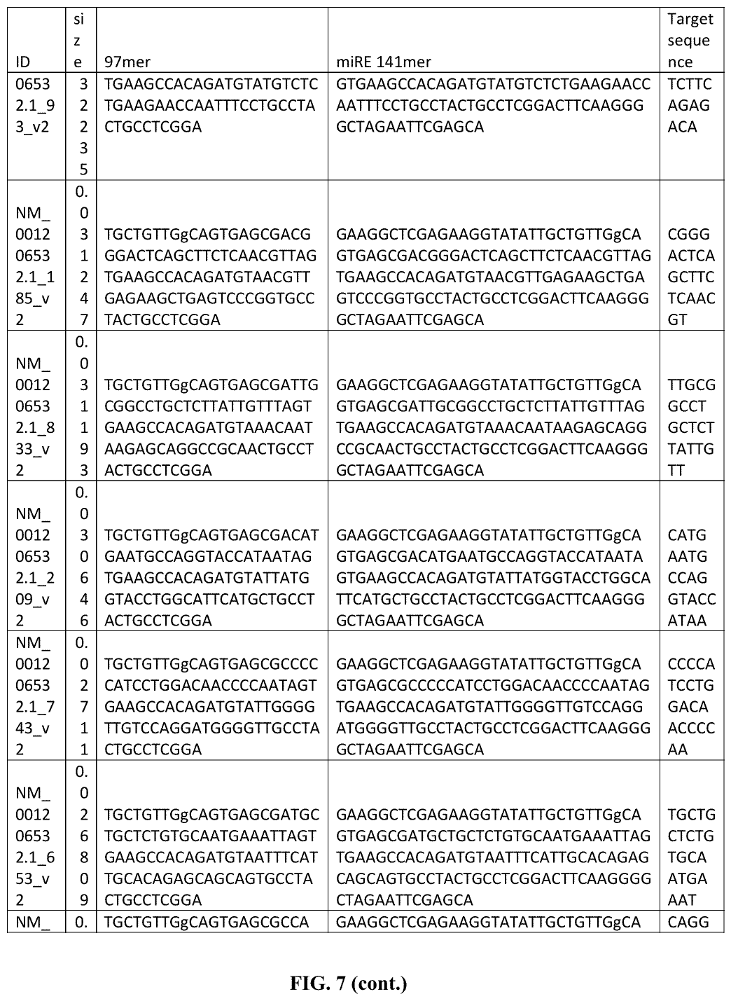

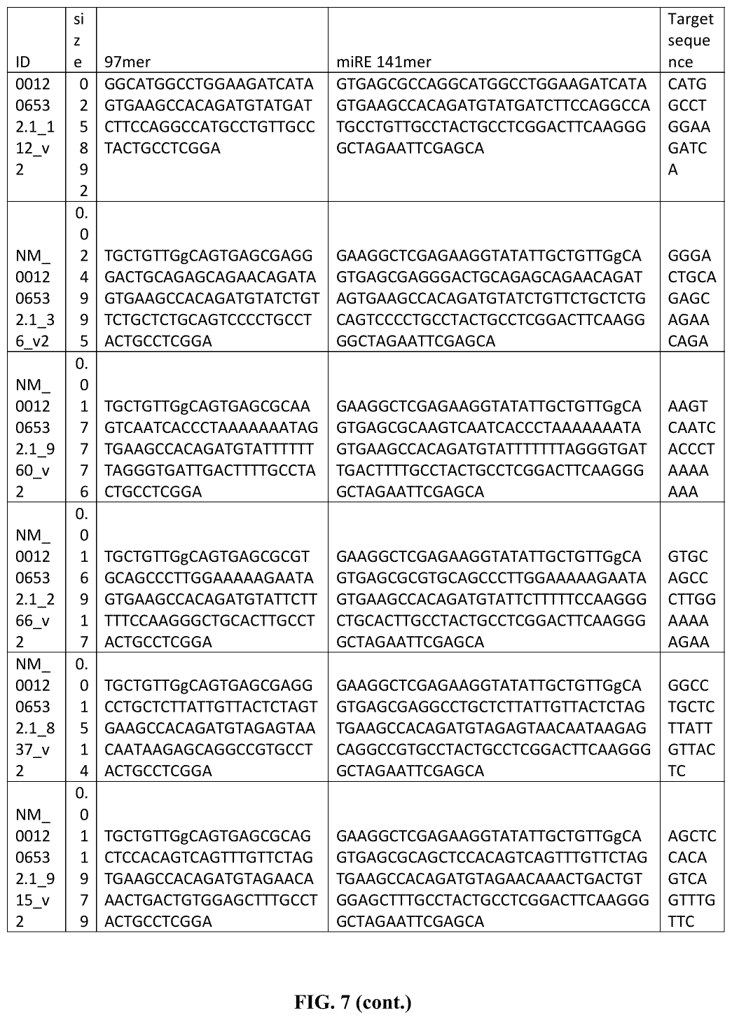

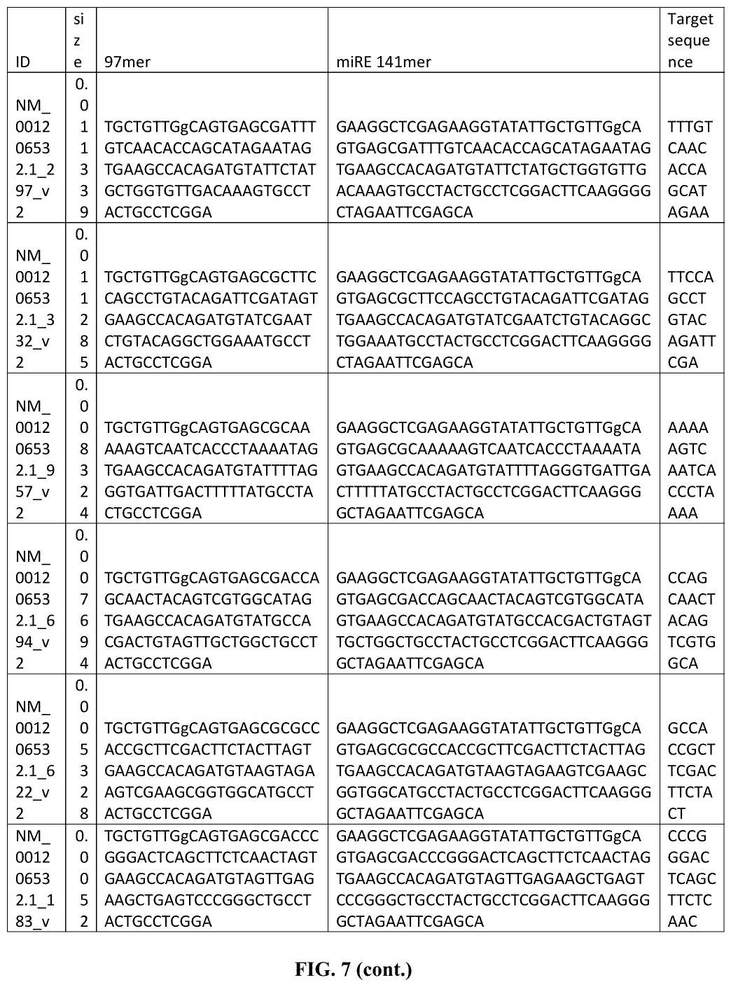

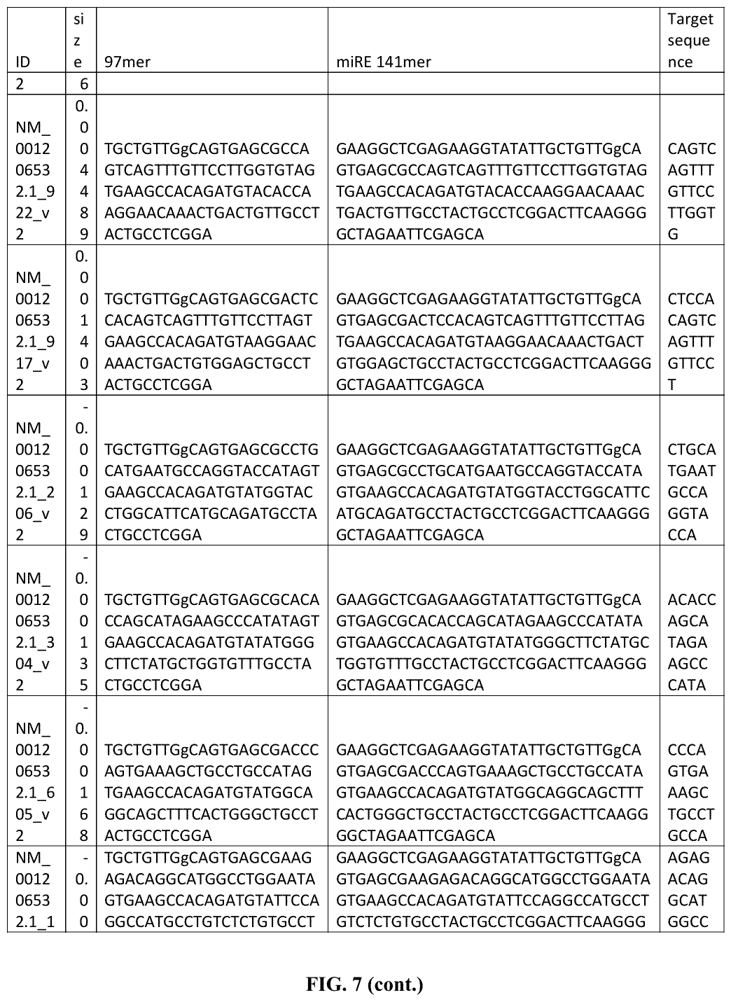

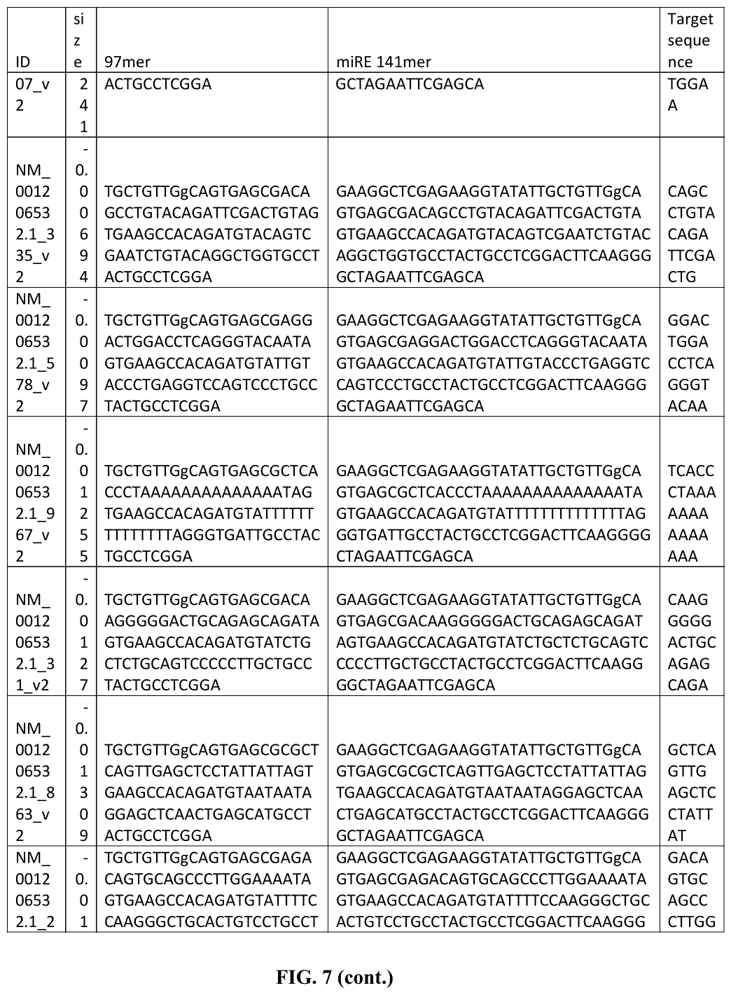

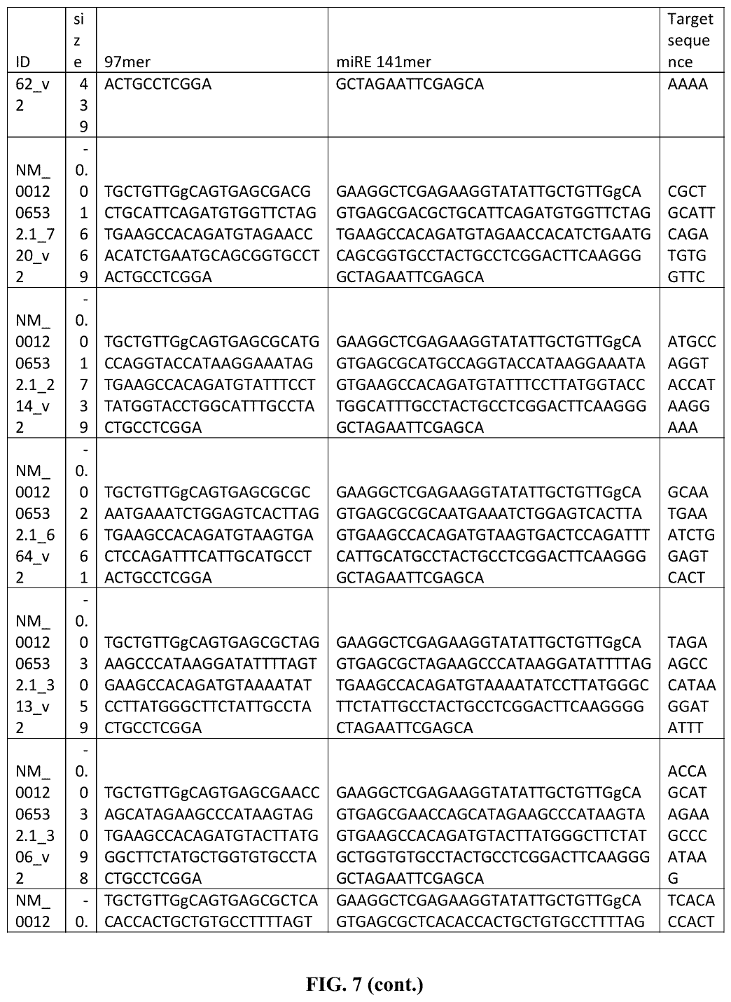

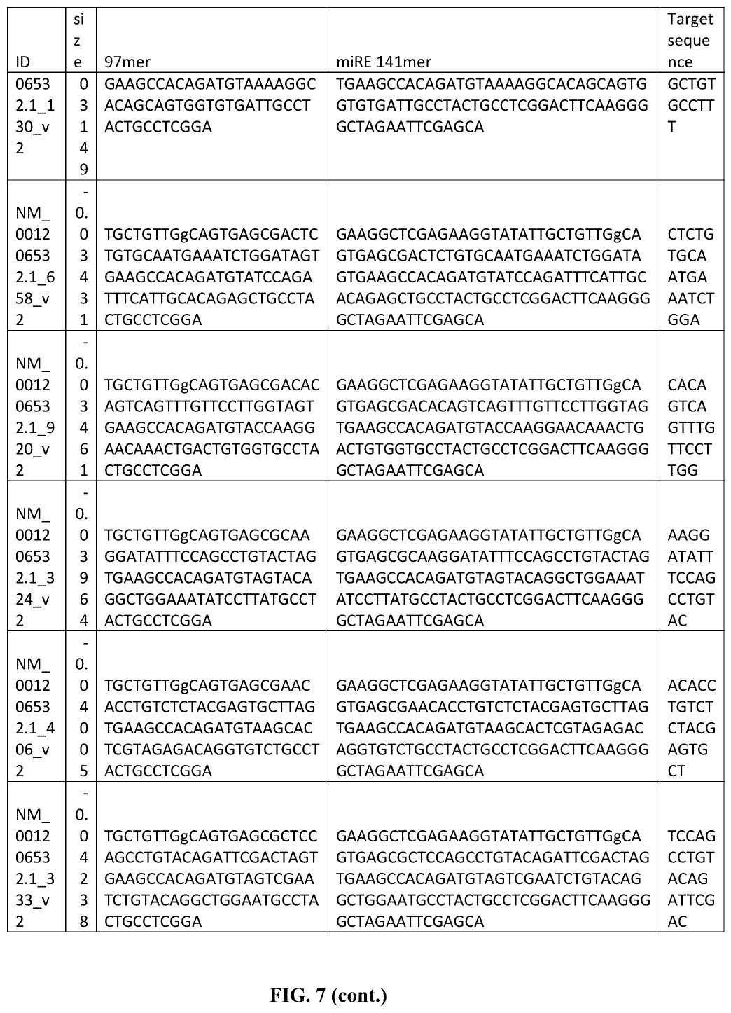

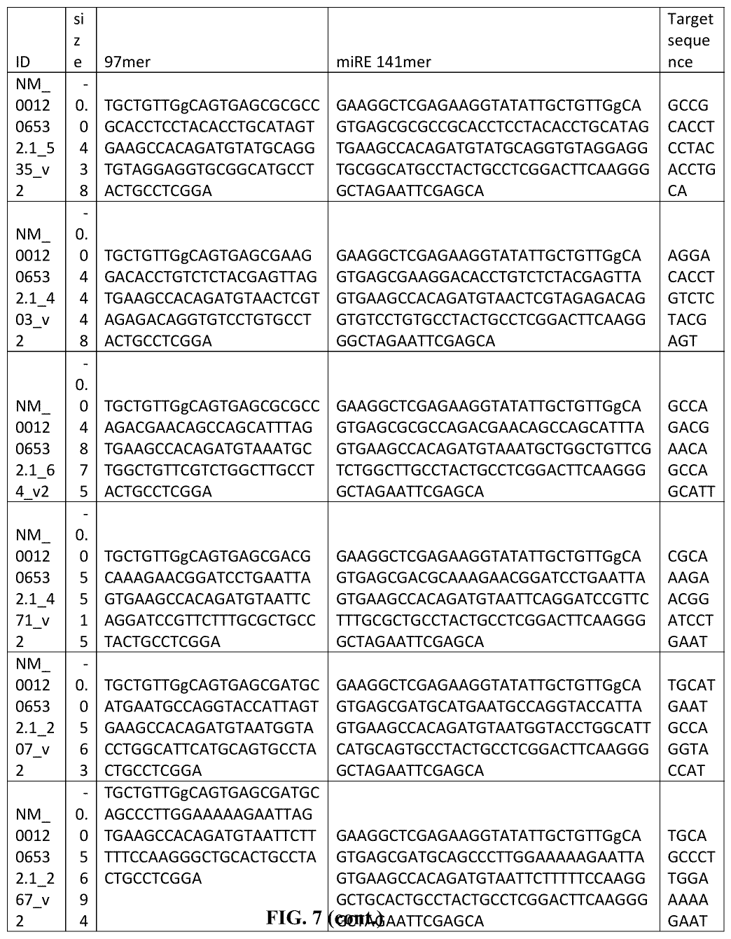

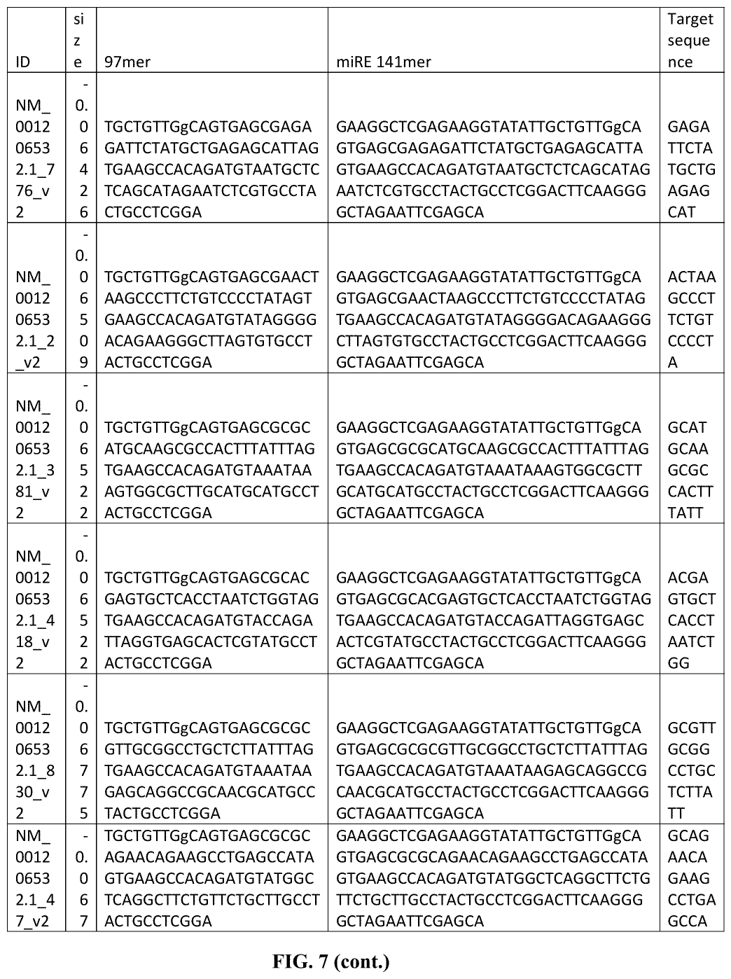

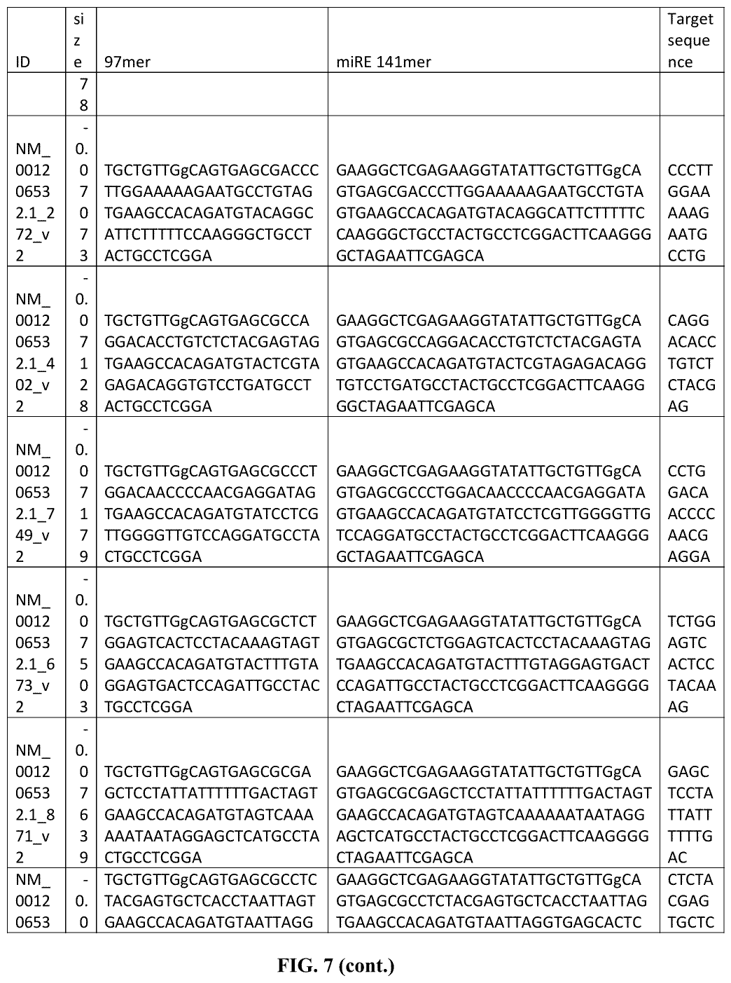

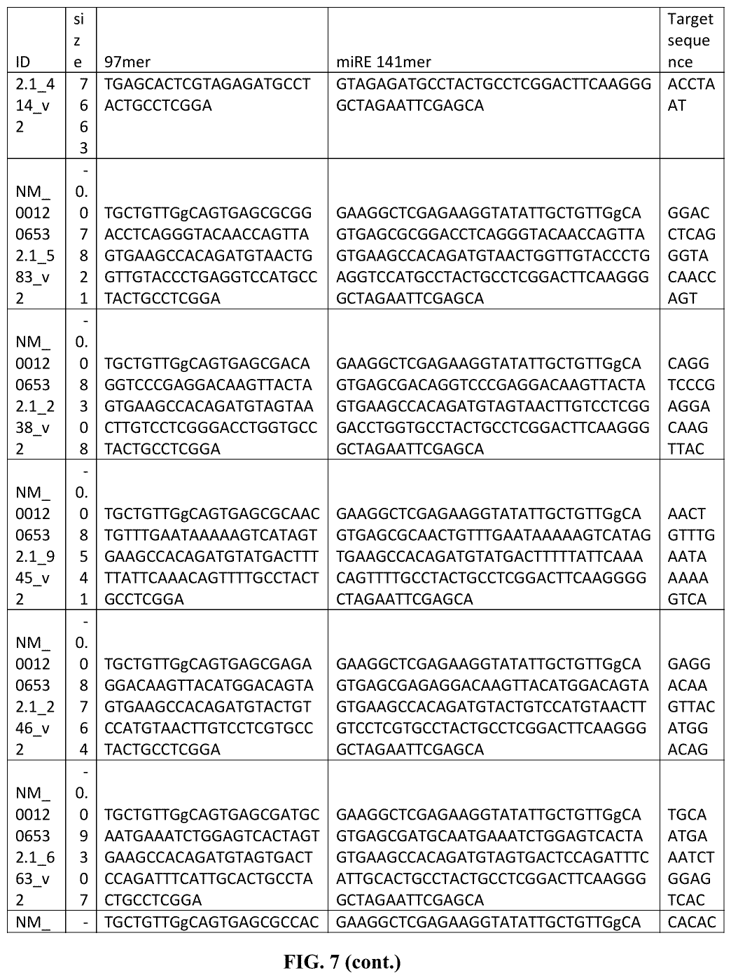

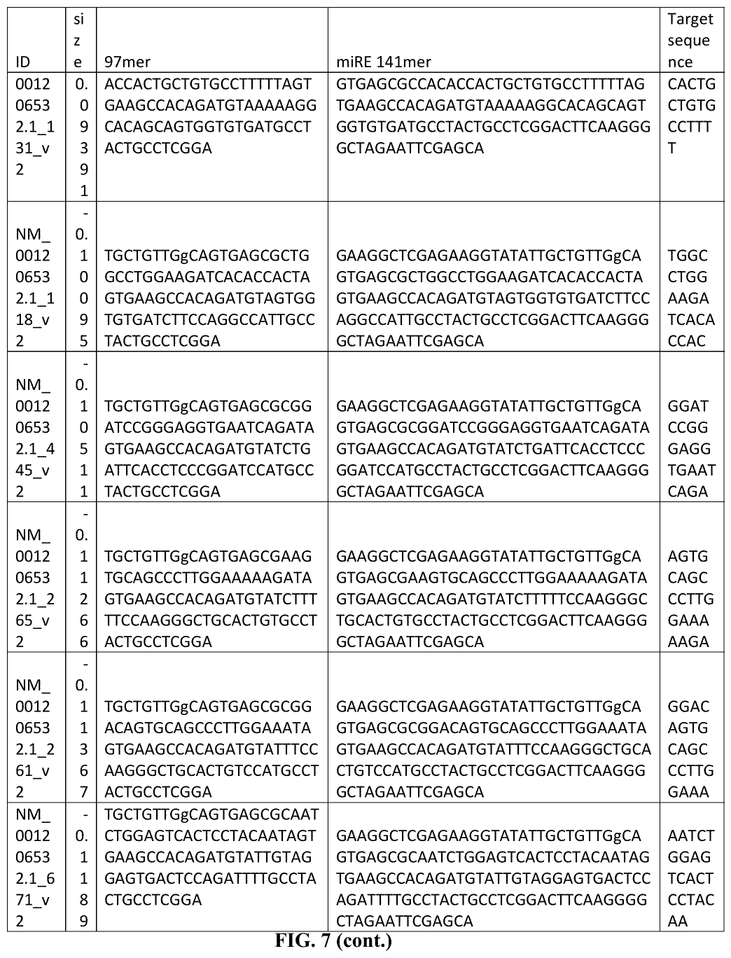

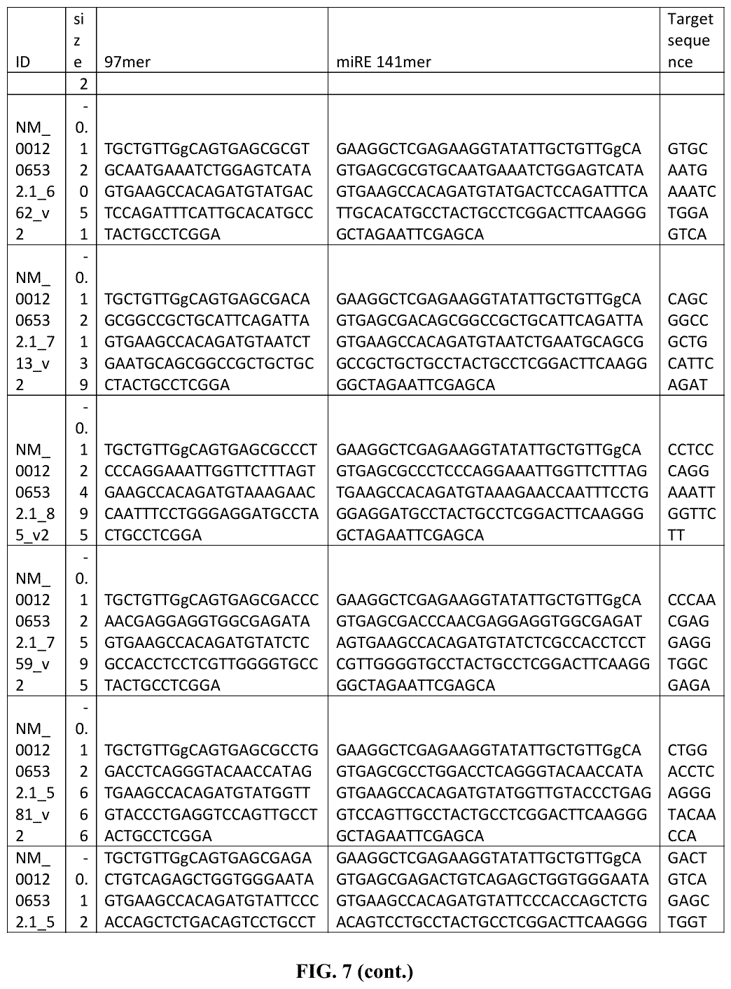

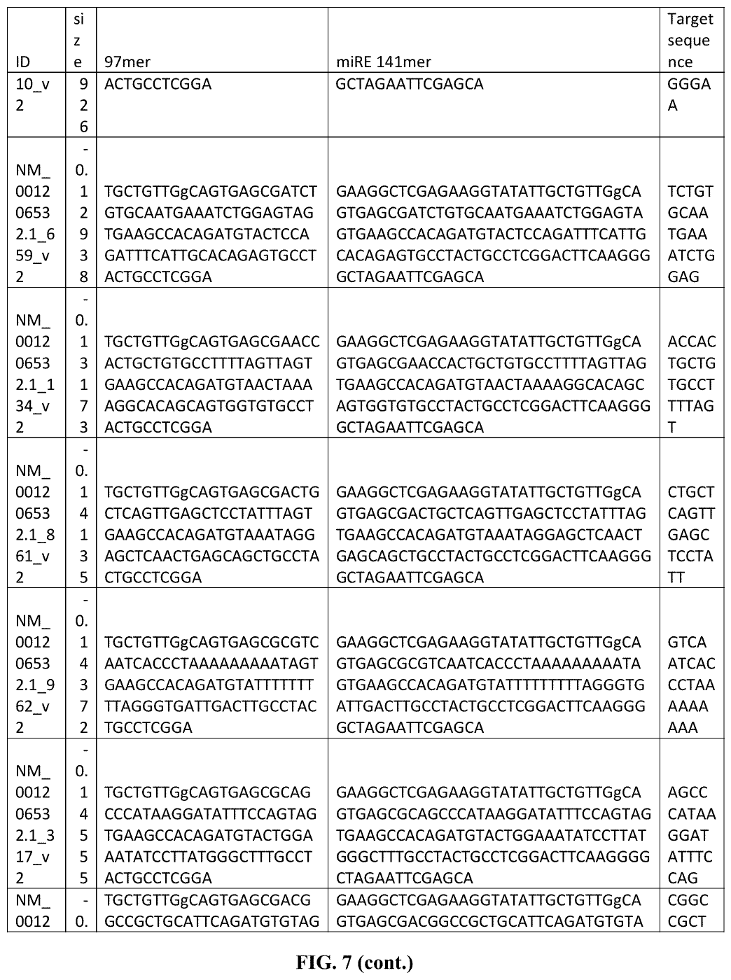

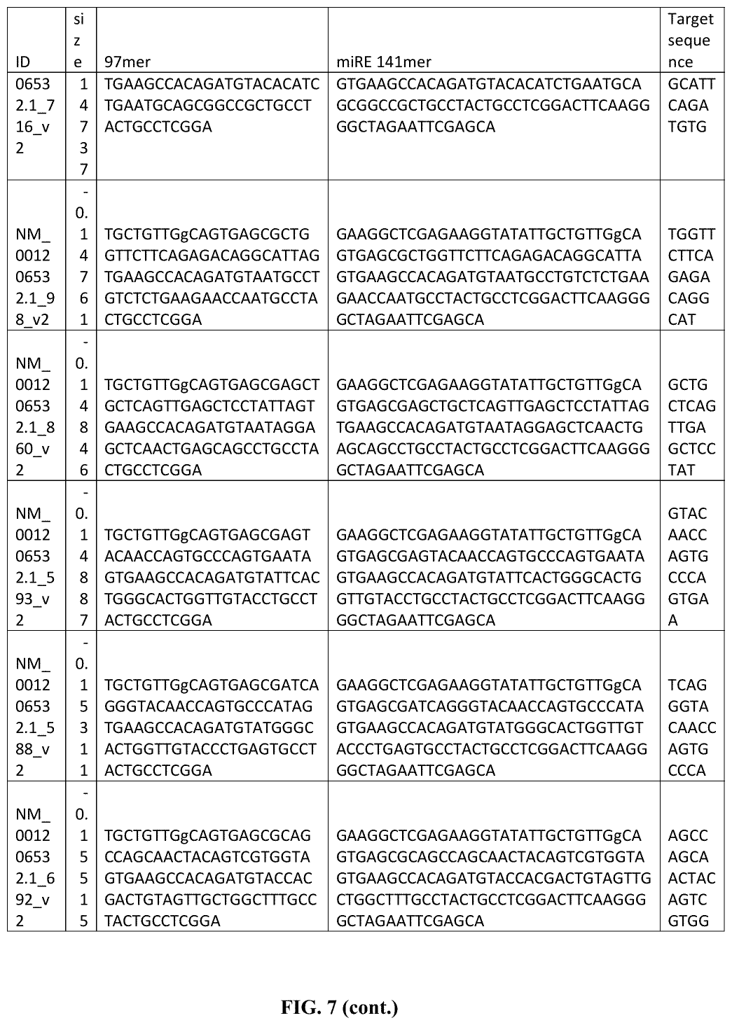

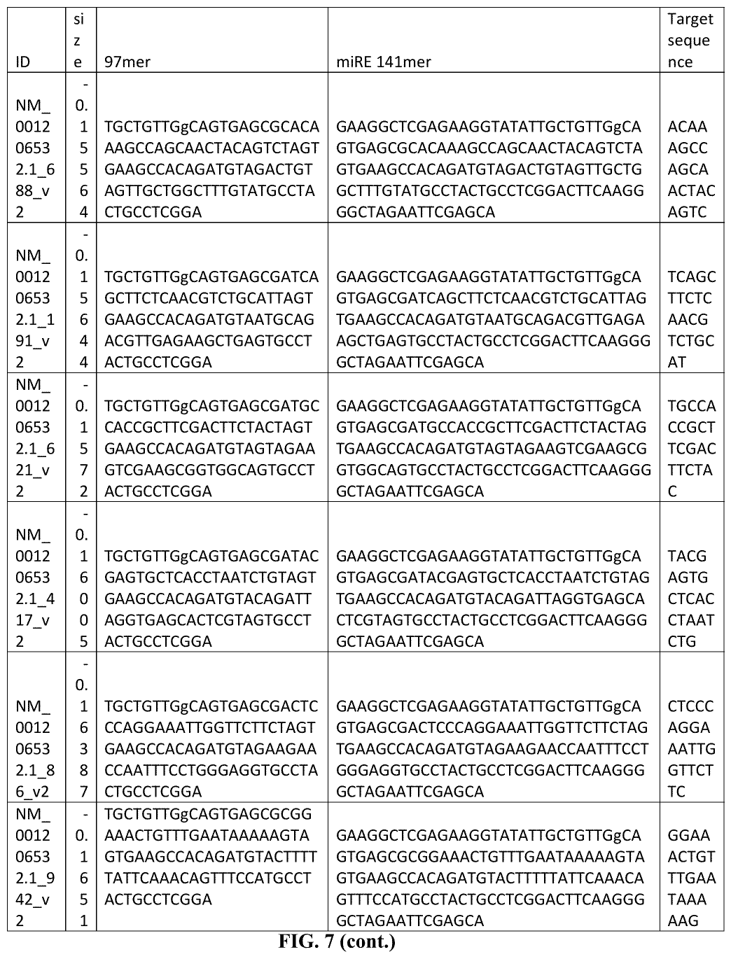

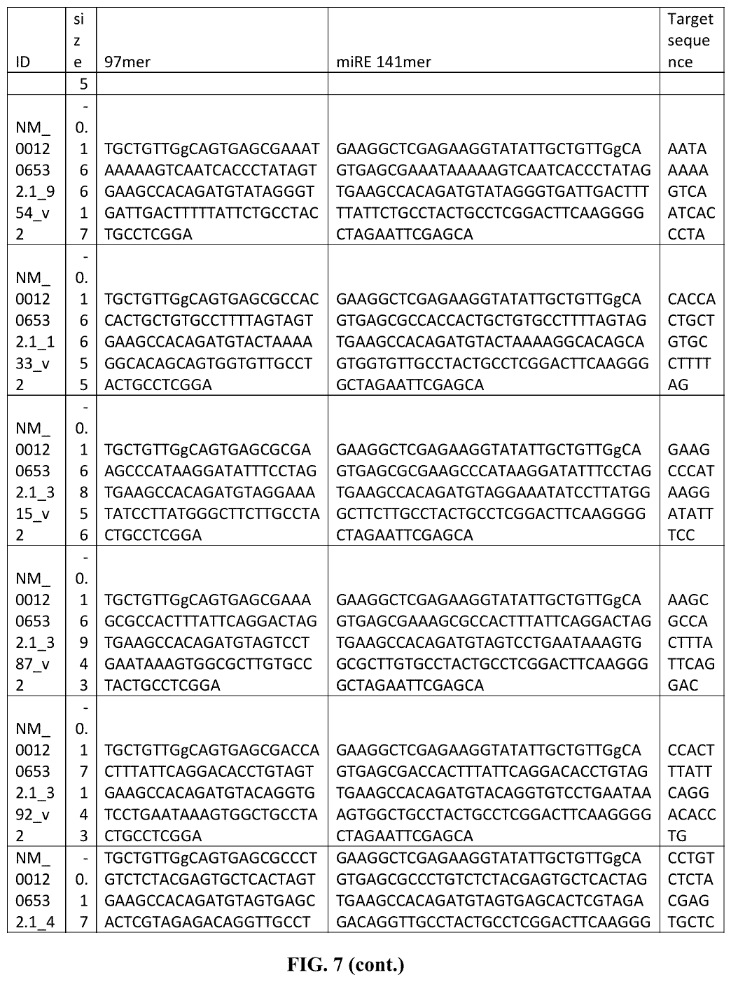

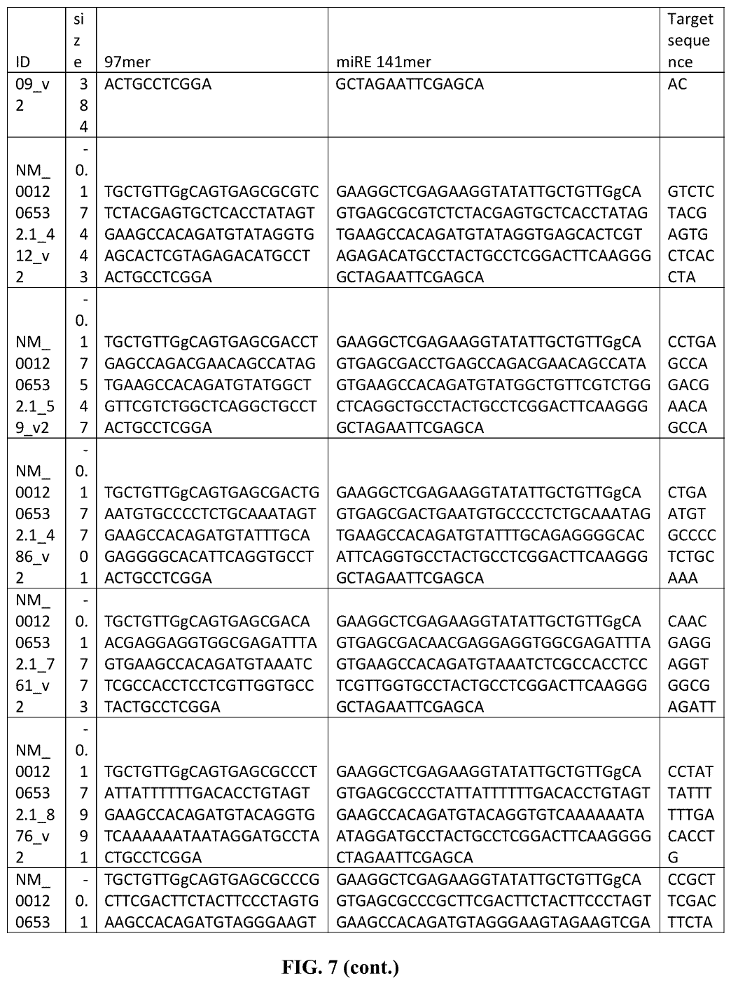

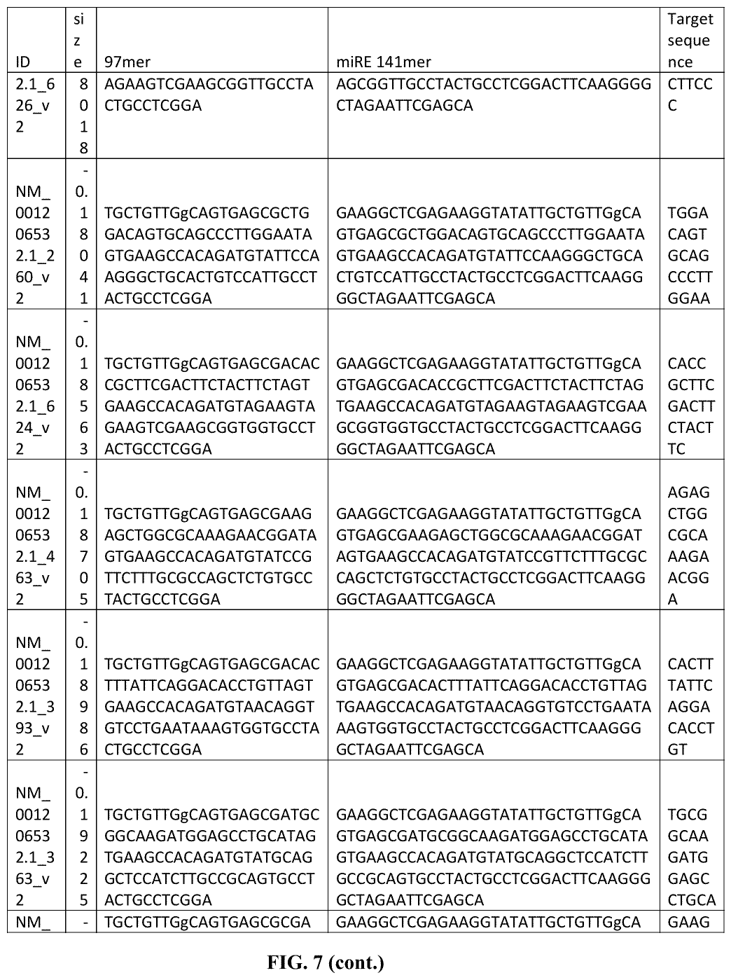

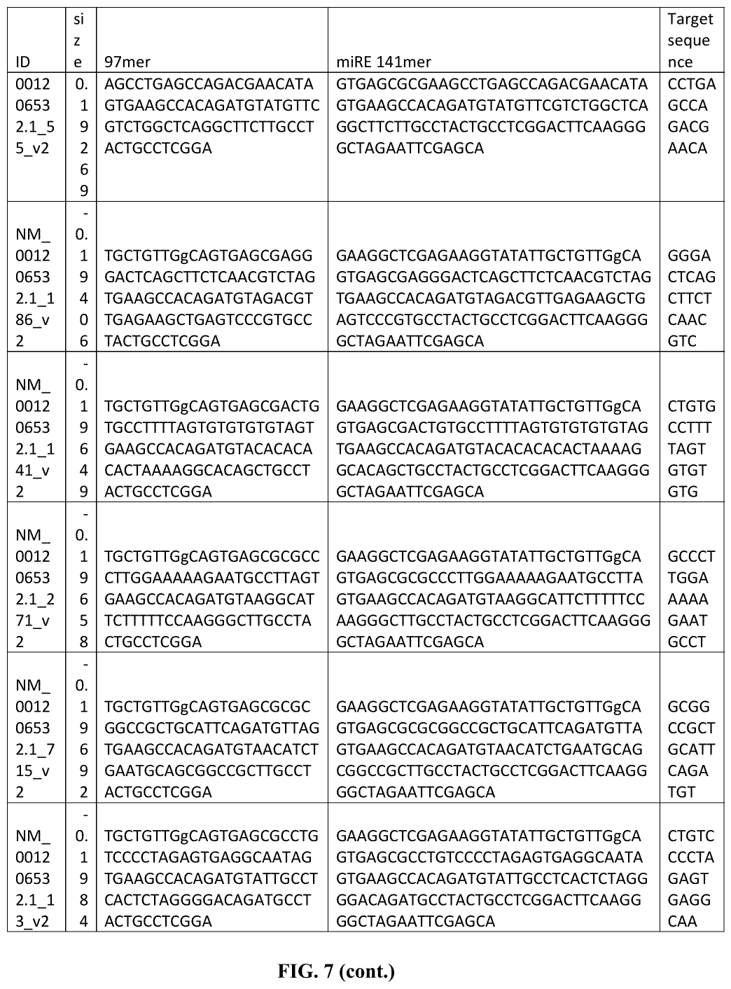

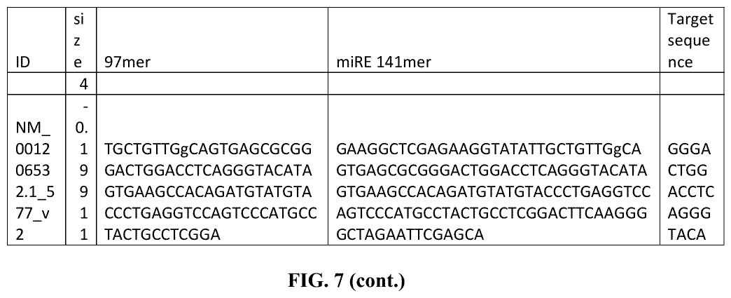

To validate the potency of predicted shRNAs 210, a functional fluorescence-based reporter assay 222 may be used. The materials required for the functional fluorescence-based reporter assay 222 include, but are not limited to: LPE-shRNAs, gBlock containing shRNA-target sites, GFP-based reporter vector, ERC Chicken Cell line, Phoenix-E Cell line, Helper plasmids: VSV-G, Gag-Pol helper plasmid (Eco or Ampho helper plasmid), Transfection reagents, Cell culture reagents (DMEM, FBS, Trypsin), Cell culture plates, Fluorescence-Activated Cell Sorter (FACS) Tubes. The list of shRNA's in the library to be tested are shown in FIG. 7.

In one embodiment, the gBlocks are double stranded DNA fragments up to about 750 bp in length (available through IDT) and the functional fluorescence-based reporter assay 222 comprises determining the about 21 bp passenger strand for each predicted shRNA; finding the full mRNA sequence of the gene of interest and mark passenger strands of predicted shRNAs, for target sites which overlap choose entire overlapping region; checking the linker sequence for the following motifs: CTCGAG (Xho1), GAATTC (EcoR1), AATAAA, and ATTAAA (Poly A Signal); and adding an Xho1 restriction site on the 5′ end and an EcoR1 restriction site to the 3′ end of the linker, and adding at least 6 random bases to each side of the linker to obtain the final “gBlock”.

In one embodiment, gBlock Cloning comprises setting up ligation using about 35 ng of digested gBlock insert (˜500 bp)+about 150 ng digested and CIP'd vector (˜6448 bp). In one embodiment, Stable Reporter Cell Line Generation comprises the following:

Day 0: Plate Plat-GP cells at 70% confluency on a 10 cm culture plate for transfection.

Day 1: Perform transfections of previously plated Plat-GP cells with 10 ug sequence verified gBlocks+VSVG (this is to keep the stable cell line “Eco-naive” so it can be infected later with virus produced using ecotropic packaging to produce single-copy integrations).

Day 2: Plate ERC Chicken cells for infections on 6-well plates.

Day 3: Harvest virus and infect ERC in 6-well plates-1:1 infection with 1 ug/mL dox (higher the infection the better, since we need the dTomato reporter to be highly expressed).

Day 3-Day 5: Incubate 2-3 days.

Day 6: Check for GFP-positive cells and expand onto 10 cm plate.

Day 6-Day 14: Continue 3-4 days of selection. Expand cells from a 6-well to a 10 cm culture plate and continue neo+dox selection. Repeat this selection process two more rounds (until 95% of the cells are GFP positive).

Day 15: Freeze down 3 vials of reporter cell line per 10 cm plate.

In one embodiment, LPE-shRNA virus production comprises the following:

Day 0: Plate Phoenix-E cells on 10 cm culture plates. Plan to have enough cells at 70% confluency for the number of shRNAs being transfected.

Day 1: Perform transfections using bug LPE-shRNA+Gag-Pol helper plasmid. Make sure appropriate control shRNAs are included.

Day 2: Plate ERC-reporter cell line harboring the shRNA-specific target sites.

Day 3: Harvest virus and infect the stable reporter cell line at 2 viral dilutions (1:8 and 1:15) to achieve infection at single-copy (5-20% mKate positive cells). Incubate cells for 2 days.

Day 5: Determine percentage of infection (%mKate) using the Guava instrument. Continue incubation of shRNA-infected reporter cells for another 4 days.

Day 8: Perform FACS using LSRII instrument (yellow-green laser will be required). Determine percentage knockdown of GFP relative to the LPE-Ren-713 control.

In one embodiment, Flow Cytometry for dTomato fluorescence determination comprises the following: Trypsinize cells by adding 1.5 ml trypsin to a 10 cm plate containing the cells; After 2 min, resuspend cells in growth medium; Transfer cells to a 15 ml Falcon tube and spin at 3000 g for 5 min; Get rid of supernantant; Wash cells with 5 ml cold PBS+2% FBS; Spin cells at 3000 g for 5 min; Get rid of supernantant and resuspend in PBS+2% FBS to desired concentration of cells; Run cells through a 40 μm cells strainer cap to prevent clogging of nozzle/instrument; and Keep cells on ice until sorting.

Nuclear Transfer to Generate Transgenic Cows

In one embodiment, a nuclear transfer to generate transgenic cows system 300 is shown in FIG. 5. The nuclear transfer to generate transgenic cows system 300 comprises using a mammary specific promoter 310 and gene editing to knockin. In one embodiment, a mammary specific promoter 310 may be used in bovine fibroblasts 320. In one embodiment, the nuclear transfer 330 to generate transgenic cows system may use CRISPR gene editing to knockin within a mammary specific gene. Methods for the somatic cell nuclear transfer technology that can be used in one embodiment to generate the cow 340 are disclosed in Kwon D J, Lee Y M, Hwang I S, Park C K, Yang B K, Cheong H T. Microtubule distribution in somatic cell nuclear transfer bovine embryos following control of nuclear remodeling type. J Vet Sci. 2010;11(2):93-101. Another method may be transgenesis, whereby the DNA is inserted into a pronucleus and integrates randomly. Silencing folate receptor expression in clones 350 specifically in the mammary glands could reduce or eliminate this antigen from milk. The cows may require supplementation with high doses of folinic acid.

To limit shRNA expression to the lactating mammary gland, the constitutive promoter used in vitro may be replaced with a murine whey acidic protein (WAP) promoter.

The Inducible CRISPR/Cas9 and RNAi method 100 described in PCT application serial no. PCT/US2016/051992, herein incorporated by reference in its entirety, is the novel combination of specific gene editing events (via CRISPR/Cas9, zinc fingers, TALENs, etc.) and RNA interference to be used sequentially and/or in combination in the same biological system (or organism or animal model).

Folate Receptor Knockdown in Cattle.

To knock down the folate receptor in cattle, the steps in Jabed A, Wagner S, McCracken J, Wells D N, Laible G. Targeted microRNA expression in dairy cattle directs production of β-lactoglobulin-free, high-casein milk. Proc Natl Acad Sci USA. 2012; 109(42):16811-6 may be used in one embodiment. The donor cell line (bovine fetal fibroblasts), previously validated for nuclear transfer (NT), was transfected with an expression cassette for shRNA. Two expanded cell clones were karyotyped and need to have normal chromosome numbers. Transgene copy numbers for the two transgenic cell lines were determined from Southern blot analyses to be approximately 30 for one and more than 200 for another.

Cell line 312/3 and the transfer of cloned embryos to recipients should result in pregnancies. One of the pregnancies may be terminated to recover the fetus and derive rejuvenated cells to preserve the unique genetics and provide future options. Of the remaining pregnancies, one resulted in the production of a live female calf.

To obtain milk and evaluate miRNA-mediated knockdown of Folate Receptor expression, we hormonally induced the transgenic calf into lactation. Analysis of the milk samples by SDS-gel electrophoresis and Coomassie blue staining revealed that none of the milk samples from the shRNAcalf contained detectable levels of Folate Receptor. By contrast, Folate Receptor should be readily detectable as a major milk protein in all WT controls, including colostrum and natural and induced milk samples. A more sensitive analysis of folate receptor levels by Western blot may be confirmed the highly effective knockdown, as all milk samples from the transgenic calf were devoid of any detectable folate receptor. To further quantify folate receptor levels and determine any effects the knockdown of folate receptor may have on milk protein composition, the milk samples may be quantified for all major milk proteins by HPLC. Consistent with the Coomassie blue staining and Western results, HPLC analysis should not detect any folate receptor antigens in the transgenic calf milk samples and confirmed comprehensive knockdown of folate receptor. The absence of the folate receptor antigen may have a strong, compensatory effect on the levels of other milk proteins. In comparison with natural and induced WT samples, all other major milk proteins may be greatly increased in the milk produced by the transgenic calf, in particular the caseins.

Cohort Expansion and Milk Analyses

Generally speaking, the Cohort expansion and milk analyses system 400 is shown in FIG. 6.

In vivo fertilization to increase cow cohort size may be used. Analytical composition testing of milk is performed at the lab near the farm.

Animal Welfare Evaluation: All transgenic animals generated at China Agricultural University have lived healthy and active lives. No physical difference between transgenic and non-transgenic animals should be observed although some abnormalities may be present (no tails).

The FRF cow will express RNA only in the mammary gland and it should not accumulate in their body. It should not affect the animal's biological system and health. Secretion from the animal's system will be tested.

Transgenic milk from previously generated cows has had a positive effect on the health and disease resistance of young cows.

Experiment Design:

The aim of these examples is to produce milk devoid of folate receptor alpha (FR alpha) starting with the commercially available bovine milk. For this purpose, protein purification techniques are used to selectively remove the FR alpha protein from cow milk without altering the basic composition of the milk and without adding any extraneous chemicals or substances to the final product. The seminal approach will use the principle of affinity chromatography to selectively remove the FR alpha from milk

This example be accomplished by: 1) Purification of FR from bovine milk; 2) Generating a polyclonal antiserum to the milk FR alpha by immunizing New Zealand white rabbit; 3) Purifying specific antibodies to FR alpha by affinity chromatography; 4) Preparing an affinity matrix containing FR alpha antibodies covalently linked to an inert solid matrix; 5) Using the FR alpha antibody affinity matrix to capture the FR alpha from A2 milk, thus rendering the product free of FR alpha protein.

### EXAMPLE 1

### Purification of FR Alpha from Bovine Milk

Preparation of affinity matrix with covalently immobilized folic acid.

Initially a 5-10 carbon linker/spacer arm will be attached to the terminal glutamic acid residue of folic acid and subsequently the end of this spacer arm will be attached to activated Sepharose 4B beads. The matrix will be extensively washed and tested for any ligand leakage and then used to capture and purify FR alpha from milk. Approximately 2-4 purifications may be necessary to obtain pure homogeneous product that can be ultimately used for immunization purposes. The FR alpha purified will be tested by SDS-Page analysis to monitor the purity of the protein.

About 10-20 mg of the protein will be needed for immunization of 10 rabbits.

### EXAMPLE 2

### Generating a Polyclonal Antiserum to the Milk FR Alpha by Immunizing New Zealand White Rabbits

The immunization of rabbits and antiserum production may be done at a commercial facility. All testing of antiserums and purification of FR alpha specific IgG is indicated below.

Four rabbits were immunized with the purified FR alpha antigen. A test bleed was performed after 3 booster immunizations. The antibody titer is determined by direct immunoprecipitation of 3HFolic acid bound FR antigen by the antiserum.

Raw milk (not pasteurized) contains on average about 5-10 ug of FR alpha per ml. Each ml of the antiserum can quantitatively bind and remove about 10 ug of FR antigen from milk as determined previously. Based on the antibody titer, about 1 ml of the antiserum can remove all of the FR alpha protein in 1 ml of milk. More or less than 1 ml of antiserum be may used per 1 ml of milk if additional removal is necessary.

Proof of concept is obtained in these preliminary experiments. For this, about 100 to about 200 mg of total IgG from the Rabbit antiserum will be bound to Protein A matrix. This Protein A-IgG Rabbit antiserum matrix will be incubated with about 10m1 of cow milk and the milk tested at various time points to determine the minimum time needed to remove all of the FR in the milk.

This example will indicate if the strategy will be successful to yield FR-Free milk.

### EXAMPLE 3

### Purifying Specific Antibodies to FR Alpha by Affinity Chromatography

For this example, between about 500 to about 1000 mg of pure FR alpha is needed to purify antibodies against FR alpha.

### EXAMPLE 4

### Preparing an Affinity Matrix containing FR Alpha Antibodies Covalently Linked to an Inert Solid Matrix

The FR alpha antibodies will be covalently attached to an activated Sepharose matrix via a 5-10 carbon linker.

### EXAMPLE 5

### Using the FR Alpha Antibody Affinity Matrix to Capture the FR Alpha from Raw Milk, thus Rendering the Product FR Alpha Free

The milk to be depleted of FR alpha will be passed through the matrix, which will result in capture of the FR in the milk. A second pass or a third pass through the matrix may be necessary if all of the FR is not removed from the milk in the first pass. The target is an undetectable level of FR alpha in the final milk product.

Antibody Generation

Monoclonal antibodies can be prepared using hybridoma methods, such as those described by Kohler and Milstein (1975) Nature 256:495. Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. radioimmunoassay (MA); enzyme-linked immunosorbent assay (ELISA)) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

Alternatively monoclonal antibodies can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries expressing CDRs of the desired species as described (McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

This invention also encompasses bispecific antibodies that specifically recognize any animal FR. Bispecific antibodies are antibodies that are capable of specifically recognizing and binding at least two different epitopes. The different epitopes can either be within the same molecule (e.g. the same folate receptor 1) or on different molecules such that both, for example, the antibodies can specifically recognize and bind a folate receptor 1 as well as, for example, 1) an effector molecule on a leukocyte such as a T-cell receptor (e.g. CD3) or Fc receptor (e.g. CD64, CD32, or CD16) or 2) a cytotoxic agent as described in detail below.

Exemplary bispecific antibodies can bind to two different epitopes, at least one of which originates in a polypeptide of the invention. Alternatively, an anti-antigenic arm of an immunoglobulin molecule can be combined with an arm which binds to a triggering molecule on a leukocyte such as a T cell receptor molecule (e.g. CD2, CD3, CD28, or B7), or Fc receptors for IgG so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Techniques for making bispecific antibodies are common in the art (Millstein et al., 1983, Nature 305:537-539; Brennan et al., 1985, Science 229:81; Suresh et al, 1986, Methods in Enzymol. 121:120; Traunecker et al., 1991, EMBO J. 10:3655-3659; Shalaby et al., 1992, J. Exp. Med. 175:217-225; Kostelny et al., 1992, J. Immunol. 148:1547-1553; Gruber et al., 1994, J. Immunol. 152:5368; and U.S. Pat. No. 5,731,168). Antibodies with more than two valencies are also contemplated. For example, trispecific antibodies can be prepared (Tutt et al., J. Immunol. 147:60 (1991)). Thus, in certain embodiments the antibodies to FR are multispecific.

### EXAMPLES

The previous examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### REFERENCES:

Dow, L. E. et al. A pipeline for the generation of shRNA transgenic mice. Nat Protoc 7, 374-393, doi:10.1038/nprot.2011.446nprot.2011.446 [pii] (2012).

Zuber, J. et al. Toolkit for evaluating genes required for proliferation and survival using tetracycline-regulated RNAi. Nat Biotechnol 29, 79-83, doi:nbt.1720 [pii] 10.1038/nbt.1720 (2011).

Zuber, J. et al. An integrated approach to dissecting oncogene addiction implicates a Myb-coordinated self-renewal program as essential for leukemia maintenance. Genes Dev 25, 1628-1640, doi:25/15/1628 [pii] 10.1101/gad.17269211 (2011).

Fellmann, C. et al. Functional Identification of Optimized RNAi Triggers Using a Massively Parallel Sensor Assay. Mol Cell 41, 733-746, doi:S1097-2765(11)00091-8 [pii] 10.1016/j.molce1.2011.02.008 (2011).

All publications and patent applications mentioned in this specification are herein incorporated by reference to the same extent as if each individual publication or patent application is specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method to produce folate receptor free (FRF) milk comprising: removing folate receptor from a milk product using a method selected from the group consisting of: size exclusion chromatography, Glycophosphate inositol (GPI) capture column affinity chromatography, Folate Receptor binding antibody affinity column chromatography, specific digestion of the folate receptor using proteases attached to the FR binding antibody, Fractionation of the milk products to remove the FR, or Affinity chromatography using ligands that specifically bind the folate receptor in milk.

2. The method of claim 1, wherein the size exclusion chromatography comprises: using a at least a 38 kDa membrane to filter the folate receptor from milk.

3. The method of claim 2, wherein the Folate Receptor binding antibody affinity column chromatography comprises: generating an antibody to the folate receptor; immoblizing the antibody to the folate receptor in a solid matrix; and mixing a milk solution with the solid matrix and antibody to the folate receptor to selectively remove the Folate Receptor antigen from the milk solution.

4. The method of claim 3, wherein the specific digestion of the folate receptor using proteases attached to the FR binding antibody comprises: attaching a protease to a folate receptor antibody; and selectively digesting the folate receptor in a milk solution.

5. The method of claim 4, wherein the Affinity chromatography using ligands that specifically bind the folate receptor in milk comprises: using folic acid to bind the folate receptor with high affinity in a milk solution.

6. A system for folate receptor free milk to decrease rates of Autism, comprising: generating shRNAs targeting the folate receptor in a mammal, conducting nucleofection of the shRNAs, identifying the positive single clone, and conducting nuclear transfer into a mammal producing milk lacking folate receptor antigens.

7. A Knockdown Folate Receptor in cow fibroblast method comprising: identifying shRNA's in a shRNA library and validating the shRNA's in a sensor assay to find shRNAs that potently and specifically silence the folate receptor; confirming the shRNA knockdown in a fibroblast; cloning a vector containing an shRNA or concatemer of shRNAs; and inserting the vector into fibroblasts.

8. The method of claim 7, wherein the vector is a mammary specific promoter-shRNA or mammary specific promoter-shRNA-shRNA-shRNA.

9. The method of claim 7, wherein the shRNA vector is specifically inserted into a mammary specific gene.

10. The method of claim 8, wherein the sensor assay is a functional fluorescence-based reporter assay including: a plurality of LPE-shRNAs, a gBlock containing shRNA-target sites, a GFP-based reporter vector, an ERC Chicken Cell line, a Phoenix-E Cell line, a plurality of Helper plasmids selected from the group of: VSV-G, Gag-Pol helper plasmid (Eco or Ampho helper plasmid), a plurality of Transfection reagents, a plurality of Cell culture reagents (DMEM, FBS, Trypsin), a plurality of Cell culture plates, a plurality of Fluorescence-Activated Cell Sorter (FACS) Tubes.

11. The method of claim 9, wherein the shRNA library is selected from the shRNA's shown in FIG. 7.

12. The method of claim 10, further comprising generating a transgenic mammal through nuclear transfer; and using a mammary specific promoter and gene editing to knockin shRNAs that potently and specifically silence the folate receptor.

13. The method of claim 11, wherein the mammary specific promoter is used in bovine fibroblasts.

14. The method of claim 12, wherein the nuclear transfer uses CRISPR gene editing to knockin within a mammary specific gene.

15. The method of claim 13, further comprising silencing folate receptor expression in the mammary glands to reduce the folate receptor antigen in milk.

16. A milk product, comprising: a folate free milk product used to make a product selected from the group consisting of: bulk milk, bulk milk used to make cheese whether or not the milk has been pasteurized, sterilized or otherwise treated to reduce the population of microbes prior to cheese making, milk powders, milk solids, caseins, caseinates, and casein hydrolysates, pasteurized, sterilized, preserved milks including microfiltered milks, UHT milks, low fat milks, modified or enhanced milks, ice-cream or other frozen dairy based confections, fermented milk products such as yoghurt or quark, cheeses including full fat, partial de-fatted and fat-free processed cheeses, milk whey, food products enriched through the addition of milk products such as soups, milk from which potentially allergenic molecules have been removed, confections such as chocolate, carbonated milk products, including those with added phosphate and/or citrate, infant formulations which may contain full, partially de-fatted or nonfat milk together with a number of additional supplements, liquid or powdered drink mixtures, and buttermilk and buttermilk powder.
